SEQUENCE LISTING (12 sequences)

SEQ ID NO: 1 - DNA, 242 bp, Lactuca  sativa
  misc_feature (12)..(12): n is a, c, g, or t
  misc_feature (106)..(106): n is a, c, g, or t
  misc_feature (133)..(133): n is a, c, g, or t
  misc_feature (227)..(227): n is a, c, g, or t
agctgaagtggnggagaatgaatggtttaagaaaggatatgtgccacctagatttgaacaggaggatgttagtcttgctgatgtggatgctattttcaatgaagcnggggattctcctatcagctgaagtggnggagaatgaatggtttaagaaaggatatgtgccacctagatttgaacaggaggatgttagtcttgctgatgtggatgctattttcaatgaagcnggggattctcctatc

SEQ ID NO: 2 - DNA, 121 bp, Lactuca sativa
  misc_feature (12)..(12): n is a, c, g, or t
  misc_feature (14)..(14): n is a, c, g, or t
  misc_feature (18)..(18): n is a, c, g, or t
  misc_feature (67)..(67): n is a, c, g, or t
  misc_feature (103)..(103): n is a, c, g, or t
  misc_feature (111)..(111): n is a, c, g, or t
gaacacacatantnaagncaagaaattgacaatgaaaatgaacttcatgaactctctagagccccanatgggttctagaagcttcccaatgaaaagaagaccnattgtgcngctaagaacc

SEQ ID NO: 3 - DNA, 121 bp, Lactuca sativa
  misc_feature (12)..(12): n is a, c, g, or t
  misc_feature (31)..(31): n is a, c, g, or t
  misc_feature (33)..(33): n is a, c, g, or t
  misc_feature (35)..(35): n is a, c, g, or t
  misc_feature (118)..(118): n is a, c, g, or t
gatatctgtagngtcaaccatttgttcaccntntnccatcaatattggcaccttcttgtagtcagaccatttgatttcctttttgttgatgggattgacttccacaattttgtatggnata

SEQ ID NO: 4 - DNA, 121 bp, Lactuca sativa
aaatctattcatctatgtttgcattctaatttagaagtagactccctgcatttgatttgcgtgacttctcaaatagtgtcaactaatgccaaaagggtatgtgcacaatcagcatgactta

SEQ ID NO: 5 - DNA, 121 bp, Lactuca sativa
ttcctgtggatggccacacattttgggtaacgaaggcccacagaattccggattccctccgagtgaagtctcaaatatactgaactgtgttcctgatggaatacgtccaacaagatggttt

SEQ ID NO: 6 - DNA, 121 bp, Lactuca sativa
agggagaaggagagggaccttgtgggttttccatgagaggaactgagcttgtgtcaatgggatggaggaacttggacctgagttcttgttcgcttcgtcttcttgatgttgttgttcttct

SEQ ID NO: 7 - DNA, 121 bp, Lactuca sativa
gatgatttctactttcattcctaaccgttggatgtgatatcttgatacctccgcccgtacgggtctcgggtcgacaatgatcgctttcatgccatgaaactctgattttgactggccgttg

SEQ ID NO: 8 - DNA, 121 bp, Lactuca sativa
taacataacaaataattttttgctatgaatgaatacttgaggraaatgagtgatttttattcagaatgagagaaagaggagaacctgacaaagattgtaagtgaagctgtcataattgtca

SEQ ID NO: 9 - DNA, 121 bp, Lactuca sativa
  misc_feature (1)..(1): n is a, c, g, or t
  misc_feature (7)..(7): n is a, c, g, or t
  misc_feature (31)..(31): n is a, c, g, or t
  misc_feature (50)..(50): n is a, c, g, or t
  misc_feature (82)..(82): n is a, c, g, or t
  misc_feature (94)..(94): n is a, c, g, or t
  misc_feature (118)..(118): n is a, c, g, or t
  misc_feature (120)..(120): n is a, c, g, or t
ncatccnttccaaggagtccaaactaaactntgaagcttggttaagtggntaccatggtcgaattccccctcttcaggaggnactaaatcgtcnttccaccattccaaattcatcatntna

SEQ ID NO: 10 - DNA, 121 bp, Lactuca sativa
  misc_feature (18)..(18): n is a, c, g, or t
  misc_feature (25)..(25): n is a, c, g, or t
agtcaaccccatcatccnamacatntggmraaatacaaatgtcaacaaccaacaacttccgaaatggtgaagtttcgattgatcgttttgatcttcatctgggttcggaatgcaacgaaga

SEQ ID NO: 11 - DNA, 121 bp, Lactuca sativa
  misc_feature (6)..(6): n is a, c, g, or t
  misc_feature (14)..(14): n is a, c, g, or t
  misc_feature (22)..(22): n is a, c, g, or t
  misc_feature (24)..(24): n is a, c, g, or t
  misc_feature (31)..(31): n is a, c, g, or t
  misc_feature (37)..(37): n is a, c, g, or t
  misc_feature (64)..(64): n is a, c, g, or t
  misc_feature (88)..(88): n is a, c, g, or t
  misc_feature (107)..(107): n is a, c, g, or t
taactnggaatatngttgaatnantacggangcatantgttcgggtgtaaactgtcggctggtntggtagtaattaggcatgaaatanttattaaggtaatcattgnttcctaatccaact

SEQ ID NO: 12 - DNA, 121 bp, Lactuca sativa
aaagctctatcaccccttctcaaatataaaaggatttggactccctgttaaatttctcatcattgccaccagtcgttcatccatatcatcaatctctggaagaaacttgagagcatgatag

(19) United States
(12) Patent Application Publication
(10) Pub. No.: US 2022/0061247 A1
(43) Pub. Date: Mar. 3, 2022

(54) METHOD and/or COMPOSITIONS FOR LETTUCE (LACTUCA SATIVA) BREEDING and/or VARIETIES DEVELOPED THEREBY

(71) Applicant: VINDARA, INC., Durham, NC (US)

(72) Inventors: Eric Jackson, Rockford, MN (US); Kevin Klassen, Aberdeen, ID (US)

(21) Appl. No.: 17/446,040

(22) Filed: Aug. 26, 2021

Related U.S. Application Data
(60) Provisional application No. 62/706,580, filed on Aug. 26, 2020.

(51) Int. Cl. A01H 6/14; A01H 5/10; A01H 5/12

(52) U.S. Cl. CPC A01H 6/1472; A01H 5/12; A01H 5/10

(57) ABSTRACT

According to the invention, there is provided novel Lettuce cultivars Vindara 13, 16, and/or 18 which produce superior plant (canopy) diameter (cm), plant height (cm), number of leaves, and overall leaf area (cm²) plants including novel SNPs associated with those traits. This invention thus relates to the seeds of Lettuce cultivars of the invention, to the plants of Lettuce cultivars of the invention, to plant parts of Lettuce cultivars of the invention, to methods for producing a Lettuce cultivar by crossing one or more of the Lettuce cultivars of the invention with another Lettuce cultivar, and to methods for producing a Lettuce cultivars containing in its genetic material one or more backcross conversion traits, or genetic markers associated with superior plant (canopy) diameter (cm), plant height (cm), number of leaves, and overall leaf area (cm²) or transgenes and to the Lettuce cultivars, plants and plant parts produced by those methods.

### CROSS-REFERENCE TO RELATED APPLICATIONS

This application claims priority to provisional application U.S. Ser. No. 62/706,580, filed Aug. 26, 2020, which is incorporated herein by reference in its entirety.

### SEQUENCE LISTING

The instant application contains a Sequence Listing which has been submitted in ASCII format via Electronic Submission and is hereby incorporated by reference in its entirety. Said ASCII copy, created on Aug. 17, 2021, is named SEQUENCE LISTING VINDARA_ST25.txt and is 6,772 bytes in size.

### FIELD

The present invention relates to the field of plant breeding and molecular biology. In particular, this invention relates to specialty Lettuce (Lactuca sativa) plants, cultivars and varieties, including methods for making and using said Lettuce plants and compositions derived thereof. Genetic markers and novel genes associated with valuable traits in Lettuce are also disclosed.

### BACKGROUND

There are numerous steps in the development of any novel, desirable plant germplasm. Plant breeding preferably begins with the analysis and definition of problems and weaknesses of the current germplasm, the establishment of program goals, and the definition of specific breeding objectives. The next step is preferable selection of germplasm that possess the traits to meet the program goals. The goal is to combine in a single variety or hybrid an improved combination of desirable traits from the parental germplasm.

There is also a need in lettuce breeding to identify lettuce germplasm that provides valuable traits including, for example, canopy diameter, plant height, number of leaves and overall leaf area. There is also a need to develop polymorphic markers for monitoring and introgressing alleles associated with these traits, and to further develop agronomically elite lettuce lines comprising these traits for enhancing plant productivity

### SUMMARY

Provided herein, are novel agronomically elite lettuce cultivars with unique and superior traits including one or more of plant (canopy) diameter (cm), plant height (cm), number of leaves, and overall leaf area (cm²) when compared to traditional and closely related cultivars grown in the same environment. In some embodiments, this invention thus relates to the seeds of the Lettuce cultivars of the invention, to plants of the Lettuce cultivars of the invention, to plant parts of the Lettuce cultivars of the invention, to methods for producing a Lettuce cultivars produced by crossing the Lettuce cultivars of the invention with other Lettuce cultivars, and to methods for producing Lettuce cultivars containing in their genetic material one or more backcross conversion traits or transgenes and to the backcross conversion Lettuce cultivars, plants and plant parts produced by those methods.

This invention also relates to Lettuce cultivars and plant parts derived from the Lettuce cultivars of the invention, to methods for producing other Lettuce cultivars derived from Lettuce cultivars of the invention and to the Lettuce cultivars and their parts derived by the use of those methods. This invention further relates to Lettuce cultivar seeds, plants and plant parts produced by crossing the Lettuce cultivars of the invention or a backcross conversion of the cultivars of the invention with another Lettuce cultivar.

The invention further relates to products and compositions produced or purified from plants of the invention including the leaves, head, stalk, stem, bolts, flowers, seeds, and the like. Products produced form the cultivar of the invention include primarily harvested leaves for consumption but other uses for plant parts are contemplated, such as animal feed, extracts and the like.

The present invention also provides single nucleotide polymorphism (SNP) markers associated with valuable traits described herein. Breeding for Lettuce plants with these traits can be greatly facilitated by the use of marker-assisted selection. The present invention provides and includes a method for screening and selecting a Lettuce plant for use in breeding to develop etlie varieties with these traits.

The present invention provides a method of introgressing an allele into a Lettuce plant for development of lettuce varieties comprising (a) crossing at least one Lettuce plant having an allele associated with one or more of plant (canopy) diameter (cm), plant height (cm), number of leaves, and overall leaf area (cm²) as depicted in one or more of SEQ ID NOs: 1, 2, 3, 4, 5, 6, 7, 8, 9, 10 11, or 12 with at least one second Lettuce plant in order to form a population, (b) genotyping with at least one Lettuce plant in the formed population with respect to said Lettuce genomic nucleic acid marker, and (c) selecting from the population at least one Lettuce plant comprising at least one genotype corresponding to a Lettuce plant having one or more of plant (canopy) diameter (cm), plant height (cm), number of leaves, and overall leaf area (cm²). In certain embodiments, the selected plants are used for further breeding. In certain embodiments of the methods, the population formed, genotyped, and selected from can be a segregating population. The invention further provides a Lettuce plant produced by such methods. More specifically the markers and genomic sequences incorporated into the plants include one or more sequences of SEQ ID NOS 1-12. In some embodiments the plants incorporating the same include a SNP from wild-type of a G at position 51 of SEQ ID NO:1, a G at position 51 of SEQ ID NO:2, a G at position 49 of SEQ ID NO:3, a G at position 49 of SEQ ID NO:4, a G at position 50 of SEQ ID NO:5, a G at position 50 of SEQ ID NO:6, a G at position 48 of SEQ ID NO;7, a T at position 50 of SEQ ID NO:8, A G at position 50 of SEQ ID NO:9, a G at position 51 of SEQ ID NO:10, a G at position 50 of SEQ ID NO: 11, or a C at position 49 of SEQ ID NO:12 as annotated to wildtype reference sequence contigs disclosed herein. Plant cultivars incorporating these marker positions or sequences are also contemplated herein.

The invention further provides a method of introgressing an allele associated with one or more of plant (canopy) diameter (cm), plant height (cm), number of leaves, and overall leaf area (cm²) that is significantly different from typical wild type lettuce into a Lettuce plant comprising: (a) crossing at least one Lettuce plant having with at least one Lettuce plant having no such traits to form a population; (b) screening the population with at least one nucleic acid marker to determine if one or more Lettuce plants from the population contains said allele, wherein the allele is selected from the group of SEQ ID NOs: 1-12. In certain embodiments of this method, the population formed, genotyped, and selected from can be a segregating population. The invention provides a Lettuce plant obtained by such methods, the Lettuce plant or variety comprising a nucleic acid molecule selected from the group of SEQ ID NOs: 1-12.

The invention provides a substantially purified nucleic acid molecule for the detection of loci related to plant (canopy) diameter (cm), plant height (cm), number of leaves, and overall leaf area (cm2) comprising a nucleic acid molecule selected from the group of SEQ ID NOs: 1-12 and complements thereof. The invention further provides assays for detecting one or more of plant (canopy) diameter (cm), plant height (cm), number of leaves, and overall leaf area (cm²) loci in a Lettuce plant.

Methods of identifying Lettuce plants comprising at least one allele associated with one or more of plant (canopy) diameter (cm), plant height (cm), number of leaves, and overall leaf area (cm²) are also provided. In certain embodiments of these methods of identifying a Lettuce plant comprising at least one allele associated with one or more of plant (canopy) diameter (cm), plant height (cm), number of leaves, and overall leaf area (cm²) in a Lettuce plant, the methods comprise: (a) genotyping at least one Lettuce plant with at least one Lettuce genomic nucleic acid marker selected from the group of SEQ ID NOs: 1-12, and (b) selecting at least one Lettuce plant comprising an allele of at least one of the nucleic acid markers that is associated with one or more of plant (canopy) diameter (cm), plant height (cm), number of leaves, and overall leaf area (cm²). In certain embodiments, the at least one Lettuce plant genotyped in step (a) and/or the at least one Lettuce plant selected in step (b) is a Lettuce plant from a population generated by a cross. In certain embodiments, the selected one or more Lettuce plants exhibit one or more of superior plant (canopy) diameter (cm), plant height (cm), number of leaves, and overall leaf area (cm²). In embodiments where the population is generated by a cross, the cross can be of at least one Lettuce plant having one or more of plant (canopy) diameter (cm), plant height (cm), number of leaves, and overall leaf area (cm²) with at least Lettuce plant having no superior plant (canopy) diameter (cm), plant height (cm), number of leaves, and overall leaf area (cm²). In still other embodiments, the methods can further comprise the step (c) of assaying the selected Lettuce plant for one or more of plant (canopy) diameter (cm), plant height (cm), number of leaves, and overall leaf area (cm²). In still other embodiments, the methods can further comprise the step of crossing the Lettuce plant selected in step (b) to another Lettuce plant. In still other embodiments, the methods can further comprise the step of obtaining seed from the Lettuce plant selected in step (b).

Also provided herein are Lettuce plants obtained by any of these methods of identifying Lettuce plants comprising at least one allele associated with one or more of plant (canopy) diameter (cm), plant height (cm), number of leaves, and overall leaf area (cm²). In certain embodiments, Lettuce plants obtained by these methods can comprise an allele of at least one nucleic acid molecule selected from the group of SEQ ID NOs: 1-12 that is associated with one or more of plant (canopy) diameter (cm), plant height (cm), number of leaves, and overall leaf area (cm²), and wherein the Lettuce plant exhibits one or more of plant (canopy) diameter (cm), plant height (cm), number of leaves, and overall leaf area (cm²). In certain embodiments, Lettuce plants obtained by these methods are elite Lettuce plants.

Methods of introgressing a one or more of plant (canopy) diameter (cm), plant height (cm), number of leaves, and overall leaf area (cm²) locus into a Lettuce plant are also provided. In certain embodiments, these methods of introgressing a one or more of plant (canopy) diameter (cm), plant height (cm), number of leaves, and overall leaf area (cm²) locus into a Lettuce plant comprise: (a) screening a population with at least one nucleic acid marker to determine if one or more Lettuce plants from the population contains a one or more of plant (canopy) diameter (cm), plant height (cm), number of leaves, and overall leaf area (cm²) locus, and (b) selecting from the population at least one Lettuce plant comprising an allele of the marker associated with the one or more of plant (canopy) diameter (cm), plant height (cm), number of leaves, and overall leaf area (cm²) locus. In certain embodiments of these methods, at least one of the markers is as provided in Table 5. In certain embodiments of these methods, at least one of the markers is located within 5 cM, 2 cM, or 1 cM of the one or more of plant (canopy) diameter (cm), plant height (cm), number of leaves, and overall leaf area (cm²). In certain embodiments of these methods, at least one of the markers is located within 100 Kb of the one or more of plant (canopy) diameter (cm), plant height (cm), number of leaves, and overall leaf area (cm²) locus. In other embodiments, at least one of the markers is located within 1 Mb, or 1 Kb of the one or more of plant (canopy) diameter (cm), plant height (cm), number of leaves, and overall leaf area (cm²).

In certain embodiments of these methods, the population is a segregating population. In certain embodiments of these methods, at least one of the markers exhibits a LOD score of greater than 2.0 with the one or more of plant (canopy) diameter (cm), plant height (cm), number of leaves, and overall leaf area (cm²) locus. In other embodiments, at least one of the markers exhibits a LOD score of greater than 3.0 or greater than 4.0 with the one or more of plant (canopy) diameter (cm), plant height (cm), number of leaves, and overall leaf area (cm²) locus. In certain embodiments of these methods, at least one of the markers is selected from the group of SEQ ID NOs: 1-12.

Also provided herein are Lettuce plants obtained by any of these methods of introgressing a one or more of plant (canopy) diameter (cm), plant height (cm), number of leaves, and overall leaf area (cm²) locus into a Lettuce plant. In certain embodiments, a Lettuce plant obtained by these methods can comprise an allele of at least one of nucleic acid marker selected from the group of SEQ ID NOs: 1-12 that is associated with one or more of plant (canopy) diameter (cm), plant height (cm), number of leaves, and overall leaf area (cm²). In certain embodiments, a Lettuce plant obtained by these methods can exhibit one or more of plant (canopy) diameter (cm), plant height (cm), number of leaves, and overall leaf area (cm²) In certain embodiments, the progeny of a Lettuce plant obtained by these methods.

Also provided are isolated nucleic acid molecules for detecting a molecular marker representing a polymorphism in Lettuce DNA, wherein the nucleic acid molecule comprises at least 15 nucleotides that include or are adjacent to the polymorphism, wherein the nucleic acid molecule is at least 70%, 80%, 90%, 95%, 98%, or 99% identical to a sequence of the same number of consecutive nucleotides in either strand of DNA that include or are adjacent to the polymorphism, and wherein the molecular marker is selected from the group of SEQ ID NOs: 1-12. In some embodiments, the polymorphism is a G at position 51 of SEQ ID NO:1, a G at position 51 of SEQ ID NO:2, a G at position 49 of SEQ ID NO:3, a G at position 49 of SEQ ID NO:4, a G at position 50 of SEQ ID NO:5, a G at position 50 of SEQ ID NO:6, a G at position 48 of SEQ ID NO;7, a T at position 50 of SEQ ID NO:8, A G at position 50 of SEQ ID NO:9, a G at position 51 of SEQ ID NO:10, a G at position 50 of SEQ ID NO: 11, or a C at position 49 of SEQ ID NO:12 as annotated to wildtype reference sequence contigs disclosed herein. In some embodiments, isolated nucleic acid molecules comprising SEQ ID NO: 1-12, or a nucleotide sequence having at least 70%, 80%, 90%, 95%, 98%, or 99% sequence identity to SEQ ID NO: 1-12 that include a G at position 51 of SEQ ID NO:1, a G at position 51 of SEQ ID NO:2, a G at position 49 of SEQ ID NO:3, a G at position 49 of SEQ ID NO:4, a G at position 50 of SEQ ID NO:5, a G at position 50 of SEQ ID NO:6, a G at position 48 of SEQ ID NO;7, a T at position 50 of SEQ ID NO:8, A G at position 50 of SEQ ID NO:9, a G at position 51 of SEQ ID NO:10, a G at position 50 of SEQ ID NO: 11, or a C at position 49 of SEQ ID NO:12 as annotated to wildtype reference sequence contigs disclosed herein are provided. In at least some embodiments the nucleic acid includes one or more base changes so that the sequence is not the naturally occurring sequence. In certain embodiments, the nucleic acids can further comprise a detectable label or provide for incorporation of a detectable label. In certain embodiments, the nucleic acid molecule hybridizes to at least one allele of the molecular marker under stringent hybridization conditions.

### DESCRIPTION OF THE FIGURES

FIGS. 1A-1E. Comparison of F4 derived Recombinant Inbred Lines (Rlls) from three cross to the corresponding F1 hybrids (●), maternal parents {@) and paternal parents (o) for number of leaves (FIG. 1A), leaf length (FIG. 1B), plant diameter (FIG. 1C), plant height (FIG. 1D), and plant area (FIG. 1E).

### DETAILED DESCRIPTION

### Definitions

In the description and tables which follow, a number of terms are used. In order to provide a clear and consistent understanding of the present invention, the following definitions are provided:

The invention provides Lettuce plants. As used herein, the term “plant” refers to plants in the genus of Lettuce and plants derived thereof. Such as Lettuce plants produced via asexual reproduction and via seed production.

The invention provides plant parts. As used herein, the term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”. Plant part may also include certain extracts such as kief or hash which includes Lettuce trichomes or glands.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, a “landrace” refers to a local variety of a domesticated plant species which has developed largely by natural processes, by adaptation to the natural and cultural environment in which it lives. The development of a landrace may also involve some selection by humans but it differs from a formal breed which has been selectively bred deliberately to conform to a particular formal, purebred standard of traits.

The invention provides plant cultivars. As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

“Elite line” means any line that has resulted from breeding and selection for superior agronomic performance. An “elite population” is an assortment of elite individuals or lines that can be used to represent the state of the art in terms of agronomically superior genotypes of a given crop species developed through breeding and selection. Similarly, an “elite germplasm” or elite strain of germplasm is an agronomically superior germplasm developed through breeding and selection.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells that can be cultured into a whole plant.

As used herein, the term “inbreeding” refers to the production of offspring via the mating between relatives. The plants resulting from the inbreeding process are referred to herein as “inbred plants” or “inbreds.”

The term LOQ as used herein refers to the limit of quantitation for Gas Chromatography (GC) and High Performance Liquid Chromatography measurements.

The term secondary metabolites as used herein refers to organic compounds that are not directly involved in the normal growth, development, or reproduction of an organism. In other words, loss of secondary metabolites does not result in immediate death of said organism.

The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

“Allele” refers to an alternative nucleic acid sequence at a particular locus; the length of an allele can be as small as 1 nucleotide base but is typically larger. For example, a first allele can occur on one chromosome, while a second allele occurs on a second homologous chromosome, e.g., as occurs for different chromosomes of a heterozygous individual, or between different homozygous or heterozygous individuals in a population. A favorable allele is the allele at a particular locus that confers, or contributes to, an agronomically desirable phenotype, or alternatively, is an allele that allows the identification of plants that can be removed from a breeding program or planting. A favorable allele of a marker is a marker allele that segregates with the favorable phenotype, or alternatively, segregates with an unfavorable plant phenotype, therefore providing the benefit of identifying plants having the unfavorable phenotype. A favorable allelic form of a chromosome interval is a chromosome interval that includes a nucleotide sequence that contributes to superior agronomic performance at one or more genetic loci physically located on the chromosome interval. “Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

“Locus” a chromosome region where a polymorphic nucleic acid, trait determinant, gene or marker is located. The loci of this invention comprise one or more polymorphisms in a population; i.e., alternative alleles are present in some individuals. A “gene locus” is a specific chromosome location in the genome of a species where a specific gene can be found.

“Linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome. Advantageously, the two linked loci are located in close proximity such that recombination between homologous chromosome pairs does not occur between the two loci during meiosis with high frequency, e.g., such that linked loci cosegregate at least about 90% of the time, e.g., 91%, 92%, 93%, 94%, 95%, 96%, 97%, 98%, 99%, 99.5%, 99.75%, or more of the time.

“Marker Assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc. “Marker Assisted Selection” (MAS) is a process by which phenotypes are selected based on marker genotypes.

The invention provides samples. As used herein, the term “sample” includes a sample from a plant, a plant part, a plant cell, or from a transmission vector, or a soil, water or air sample.

The invention provides progeny. As used herein, the term “progeny” refers to any plant resulting from a vegetative or sexual reproduction from one or more parent plants or descendants thereof. For instance, a progeny plant may be obtained by cloning or selfing of a parent plant or by crossing two parent plants and include selfings as well as the F1 or F2 or still further generations. An F1 is a first-generation progeny produced from parents at least one of which is used for the first time as donor of a trait, while offspring of second generation (F2) or subsequent generations (F3, F4, etc.) are specimens produced from selfings of F1's F2's etc. An F1 may thus be (and usually is) a hybrid resulting from a cross between two true breeding parents (true-breeding is homozygous for a trait), while an F2 may be (and usually is) an progeny resulting from self-pollination of said F1 hybrids.

The invention provides methods for crossing a first plant with a second plant. As used herein, the term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term backcrossing is a process in which a breeder crosses progeny back to one of the parents one or more times, for example, a first generation hybrid F₁ with one of the parental genotype of the F1 hybrid.

The invention provides donor plants and recipient plants. As used herein, “donor plants” refer to the parents of a variety which contains the gene or trait of interest which is desired to be introduced into a second variety (e.g., “recipient plants”).

In some embodiments, the present invention provides methods for obtaining plant genotypes comprising recombinant genes. As used herein, the term “genotype” refers to the genetic makeup of an individual cell, cell culture, tissue, organism (e.g., a plant), or group of organisms. A “haplotype” is the genotype of an individual at a plurality of genetic loci. Typically, the genetic loci described by a haplotype are physically and genetically linked, i.e., on the same chromosome interval. The terms “phenotype,” or “phenotypic trait” or “trait” refers to one or more trait of an organism. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, i.e., a “single gene trait.” In other cases, a phenotype is the result of several genes. “Phenotype” means the detectable characteristics of a cell or organism which can be influenced by genotype.

“Molecular phenotype” is a phenotype detectable at the level of a population of one or more molecules. Such molecules can be nucleic acids, proteins, or metabolites. A molecular phenotype could be an expression profile for one or more gene products, e.g., at a specific stage of plant development, in response to an environmental condition or stress, etc.

A “population of plants” or “plant population” means a set comprising any number, including one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants. The population members need not be identical to the population members selected for use in subsequent cycles of analyses or those ultimately selected to obtain final progeny plants. Often, a plant population is derived from a single biparental cross, but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

In some embodiments, the present invention provides homozygotes. As used herein, the term “homozygote” refers to an individual cell or plant having the same alleles at one or more loci.

In some embodiments, the present invention provides homozygous plants. As used herein, the term “homozygous” refers to the presence of identical alleles at one or more loci in homologous chromosomal segments.

In some embodiments, the present invention provides hemizygotes. As used herein, the term “hemizygotes” or “hemizygous” refers to a cell, tissue, organism or plant in which a gene is present only once in a genotype, as a gene in a haploid cell or organism, a sex-linked gene in the heterogametic sex, or a gene in a segment of chromosome in a diploid cell or organism where its partner segment has been deleted. In some embodiments, the present invention provides heterozygotes. As used herein, the terms “heterozygote” and “heterozygous” refer to a diploid or polyploid individual cell or plant having different alleles (forms of a given gene) present at least at one locus. In some embodiments, the cell or organism is heterozygous for the gene of interest which is under control of the synthetic regulatory element.

The invention provides methods for obtaining plant lines comprising recombinant genes. As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The invention provides open-pollinated populations. As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

The invention provides self-pollination populations. As used herein, the term “self-crossing”, “self-pollinated” or “self-pollination” means the pollen of one flower on one plant is applied (artificially or naturally) to the ovule (stigma) of the same or a different flower on the same plant.

The invention provides ovules and pollens of plants. As used herein when discussing plants, the term “ovule” refers to the female gametophyte, whereas the term “pollen” means the male gametophyte.

The invention provides plant tissue. As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The invention provides methods for obtaining plants comprising recombinant genes through transformation. As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

The invention provides transformants comprising recombinant genes. As used herein, the term “transformant” refers to a cell, tissue or organism that has undergone transformation. The original transformant is designated as “T0” or “T₀.” Selfing the T0 produces a first transformed generation designated as “T1” or “T₁.”

In some embodiments, the present invention provides organisms with recombinant genes. As used herein, an “organism” refers any life form that has genetic material comprising nucleic acids including, but not limited to, prokaryotes, eukaryotes, and viruses. Organisms of the present invention include, for example, plants, animals, fungi, bacteria, and viruses, and cells and parts thereof.

“Recombinant” in reference to a nucleic acid or polypeptide indicates that the material (e.g., a recombinant nucleic acid, gene, polynucleotide, polypeptide, etc.) has been altered by human intervention. The term recombinant can also refer to an organism that harbors recombinant material, e.g., a plant that comprises a recombinant nucleic acid is considered a recombinant plant.

“Exogenous nucleic acid” is a nucleic acid that is not native to a specified system (e.g., a germplasm, plant, variety, etc.), with respect to sequence, genomic position, or both. As used herein, the terms “exogenous” or “heterologous” as applied to polynucleotides or polypeptides typically refers to molecules that have been artificially supplied to a biological system (e.g., a plant cell, a plant gene, a particular plant species or variety or a plant chromosome under study) and are not native to that particular biological system. The terms can indicate that the relevant material originated from a source other than a naturally occurring source, or can refer to molecules having a non-natural configuration, genetic location or arrangement of parts. In contrast, for example, a “native” or “endogenous” gene is a gene that does not contain nucleic acid elements encoded by sources other than the chromosome or other genetic element on which it is normally found in nature. An endogenous gene, transcript or polypeptide is encoded by its natural chromosomal locus, and not artificially supplied to the cell.

“Genetic element” or “gene” refers to a heritable sequence of DNA, i.e., a genomic sequence, with functional significance. The term “gene” can also be used to refer to, e.g., a cDNA and/or an mRNA encoded by a genomic sequence, as well as to that genomic sequence.

“Polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

“Operably linked” refers to the association of two or more nucleic acid elements in a recombinant DNA construct, e.g. as when a promoter is operably linked with DNA that is transcribed to RNA whether for expressing or suppressing a protein. Recombinant DNA constructs can be designed to express a protein which can be an endogenous protein, an exogenous homologue of an endogenous protein or an exogenous protein with no native homologue. Alternatively, recombinant DNA constructs can be designed to suppress the level of an endogenous protein, e.g. by suppression of the native gene. Such gene suppression can be effectively employed through a native RNA interference (RNAi) mechanism in which recombinant DNA comprises both sense and anti-sense oriented DNA matched to the gene targeted for suppression where the recombinant DNA is transcribed into RNA that can form a double-strand to initiate an RNAi mechanism. Gene suppression can also be effected by recombinant DNA that comprises anti-sense oriented DNA matched to the gene targeted for suppression. Gene suppression can also be effected by recombinant DNA that comprises DNA that is transcribed to a microRNA matched to the gene targeted for suppression.

“Adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “adjacent” to the polymorphism.

As used herein, “consensus sequence” refers to a constructed DNA sequence which identifies SNP and Indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

“Transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extrachromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Vector” is a polynucleotide or other molecule that transfers nucleic acids between cells. Vectors are often derived from plasmids, bacteriophages, or viruses and optionally comprise parts which mediate vector maintenance and enable its intended use. A “cloning vector” or “shuttle vector” or “subcloning vector” contains operably linked parts that facilitate subcloning steps (e.g., a multiple cloning site containing multiple restriction endonuclease sites). The term “expression vector” as used herein refers to a vector comprising operably linked polynucleotide sequences that facilitate expression of a coding sequence in a particular host organism (e.g., a bacterial expression vector or a plant expression vector).

“Plant (canopy) diameter (cm), plant height (cm), number of leaves, and overall leaf area (cm2) allele” refers to the nucleic acid sequence associated plant (canopy) diameter (cm), plant height (cm), number of leaves, and overall leaf area (cm2) in Lettuce plants at a particular locus.

“Plant (canopy) diameter (cm), plant height (cm), number of leaves, and overall leaf area (cm2) locus” refers to a locus associated with plant (canopy) diameter (cm), plant height (cm), number of leaves, and overall leaf area (cm2) in Lettuce plants.

### Lettuce

The present invention identifies previously-unknown genetic loci which confer plant (canopy) diameter (cm), plant height (cm), number of leaves, and overall leaf area (cm2), and provides novel molecular markers linked to plant (canopy) diameter (cm), plant height (cm), number of leaves, and overall leaf area (cm2) in Lettuce plants. The invention further provides methods for introgression of genetic loci conferring plant (canopy) diameter (cm), plant height (cm), number of leaves, and overall leaf area (cm2) into plant varieties previously lacking such loci. The genetic loci, markers, and methods provided by the invention therefore represent a significant advance in the art, enabling production of new varieties with valuable traits.

In some embodiments, the invention therefore provides quantitative trait loci (QTL) that demonstrate significant co-segregation with plant (canopy) diameter (cm), plant height (cm), number of leaves, and overall leaf area (cm2). The QTL of the invention can be tracked during plant breeding or introgressed into a desired genetic background in order to provide novel plants exhibiting plant (canopy) diameter (cm), plant height (cm), number of leaves, and overall leaf area (cm2) and one or more other beneficial traits. In particular embodiments, the invention identifies for the first time a locus on chromosome 4 of the Lettuce genome, which is associated with plant (canopy) diameter (cm), plant height (cm), number of leaves, and overall leaf area (cm2). In some embodiments, the Lettuce cultivars of the invention comprise at least one polymorphism selected from an ‘A’ at position 43581285, a ‘T’ at position 43581290, and an ‘A’ at position 43581292 with reference to the position numbering of chromosome 4 (CM011608.1).

In other embodiments, the invention provides molecular markers linked to the QTL of the invention and methods of using the markers for detection of and selection for plant (canopy) diameter (cm), plant height (cm), number of leaves, and overall leaf area (cm2). Embodiments of the invention therefore include specific markers, chromosome intervals comprising the markers, and methods of detecting markers genetically linked to the locus on chromosome 4 to identify plant lines with favorable trait. In certain embodiments, the invention further provides markers closely genetically linked to SEQ ID NOs: 1-12, and chromosome intervals whose borders include such markers. Also provided herein are markers that are useful for detecting the presence or absence of plant (canopy) diameter (cm), plant height (cm), number of leaves, and overall leaf area (cm2) alleles within the QTL of the invention that can be used in marker assisted selection (MAS) breeding programs to produce plants with plant (canopy) diameter (cm), plant height (cm), number of leaves, and overall leaf area (cm2).

The invention further provides methods of using the markers identified herein to introgress loci associated with plant (canopy) diameter (cm), plant height (cm), number of leaves, and overall leaf area (cm2) into plants. Thus, one skilled in the art can use the invention to create novel Lettuce plants with plant (canopy) diameter (cm), plant height (cm), number of leaves, and overall leaf area (cm2) by crossing a donor line comprising a QTL associated with plant (canopy) diameter (cm), plant height (cm), number of leaves, and overall leaf area (cm2) into any desired recipient line, with or without MAS. Resulting progeny can be selected to be genetically similar to the recipient line except for the plant (canopy) diameter (cm), plant height (cm), number of leaves, and overall leaf area (cm2) QTL.

### Quantitative Trait Loci

The term “chromosome interval” designates a contiguous linear span of genomic DNA that resides on a single chromosome. A chromosome interval may comprise a QTL linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make or practice the invention, as such an interval will segregate at meiosis as a linkage block. In accordance with the invention, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as plant (canopy) diameter (cm), plant height (cm), number of leaves, and overall leaf area (cm2), in plant populations. In some embodiments, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

In some embodiments, the invention provides a chromosomal interval comprising a QTL associated with plant (canopy) diameter (cm), plant height (cm), number of leaves, and overall leaf area (cm2). The invention also provides multiple markers associated with plant (canopy) diameter (cm), plant height (cm), number of leaves, and overall leaf area (cm2), for example any one or more of the markers having the sequence of SEQ ID NOs: 1-12. The invention therefore provides plants comprising a nucleic acid molecule selected from the group one or more of SEQ ID NOs: 1-12, fragments thereof, or complements thereof. The present invention further provides a plant comprising alleles of the chromosome interval linked to plant (canopy) diameter (cm), plant height (cm), number of leaves, and overall leaf area (cm2) or fragments and complements thereof as well as any plant comprising any combination of one or more plant (canopy) diameter (cm), plant height (cm), number of leaves, and overall leaf area (cm2) loci selected from the group consisting of SEQ ID NOs: 1-12. Plants provided by the invention may be homozygous or heterozygous for such alleles.

Thus, one skilled in the art can use the invention to create novel Lettuce plants with plant (canopy) diameter (cm), plant height (cm), number of leaves, and overall leaf area (cm2) by associating trait phenotypes with genotypes at previously unknown trait loci in the Lettuce genome. Disclosed herein are chromosome intervals that comprise alleles responsible for phenotypic differences between Lettuce lines with favorable or unfavorable trait. The chromosome intervals of the invention are characterized in specific embodiments by genomic regions including the markers SEQ ID NOs: 1-12, which comprise markers closely linked to (within 20 cM of) the loci reported herein.

Examples of markers useful for this purpose comprise the SNP markers listed in Table 4, or any marker linked thereto, including a marker that maps within or is genetically linked to the chromosome intervals described herein, including the termini of the intervals. Such markers can be assayed simultaneously or sequentially in a single sample or population of samples.

Accordingly, the compositions and methods of the present invention can be utilized to guide MAS or breeding Lettuce varieties with a desired complement (set) of allelic forms of chromosome intervals associated with superior agronomic performance (plant (canopy) diameter (cm), plant height (cm), number of leaves, and overall leaf area (cm2), along with any other available markers for yield, disease tolerance, etc.). Any of the disclosed marker alleles can be introduced into a Lettuce line via introgression, by traditional breeding (or introduced via transformation, or both) to yield a Lettuce plant with superior agronomic performance. The number of alleles associated with plant (canopy) diameter (cm), plant height (cm), number of leaves, and overall leaf area (cm2) that can be introduced or be present in a Lettuce plant of the present invention ranges from 1 to the number of alleles disclosed herein, each integer of which is incorporated herein as if explicitly recited.

MAS using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein can thus find use within the scope of this invention.

Similarly, by identifying plants lacking a desired marker locus, plants having unfavorable plant (canopy) diameter (cm), plant height (cm), number of leaves, and overall leaf area (cm2) traits can be identified and eliminated from subsequent crosses. These marker loci can be introgressed into any desired genomic background, germplasm, plant, line, variety, etc., as part of an overall MAS breeding program designed to enhance plant (canopy) diameter (cm), plant height (cm), number of leaves, and overall leaf area (cm2). The invention also provides chromosome QTL intervals that can be used in MAS to select plants that demonstrate plant (canopy) diameter (cm), plant height (cm), number of leaves, and overall leaf area (cm2). The present invention also extends to a method of making a progeny Lettuce plant and the resulting progeny Lettuce plants. The method comprises, in an embodiment, crossing a first parent Lettuce plant with a second Lettuce plant and growing the Lettuce plant parent under plant growth conditions to yield Lettuce plant progeny. Methods of crossing and growing Lettuce plants are well within the ability of those of ordinary skill in the art. Such Lettuce plant progeny can be assayed for alleles associated with plant (canopy) diameter (cm), plant height (cm), number of leaves, and overall leaf area (cm2) as disclosed herein and, thereby, the desired progeny selected. Such progeny plants or seed thereof can be sold commercially for Lettuce production, used for food or feed, processed to obtain a desired constituent of the Lettuce, or further utilized in subsequent rounds of breeding. At least one of the first or second Lettuce plants may be a Lettuce plant of the present invention in that it comprises at least one of the allelic forms of the markers of the present invention, such that the progeny are capable of inheriting the allele.

Often, a method of the present invention may be applied to at least one related Lettuce plant such as from a progenitor or descendant line in the subject Lettuce plants' pedigree such that inheritance of the desired allele can be traced. The number of generations separating the Lettuce plants being subjected to the methods of the present invention may be, in specific embodiments, from 1 to 20 or more, commonly 1 to 10, and including 1, 2, 3, 4, 5, 6, 7, 8, 9, 10 or more generations of separation, and often a direct descendant or parent of the Lettuce plant will be subject to the method (i.e., one generation of separation).

Thus, the invention permits one skilled in the art to detect the presence or absence of favorable plant (canopy) diameter (cm), plant height (cm), number of leaves, and overall leaf area (cm2) in the genomes of Lettuce plants as part of a MAS program. In one embodiment, a breeder ascertains the genotype at one or more markers for a parent having favorable plant traits, which contains a plant (canopy) diameter (cm), plant height (cm), number of leaves, and overall leaf area (cm2) allele, and the genotype at one or more markers for a parent with unfavorable trait, which lacks the plant (canopy) diameter (cm), plant height (cm), number of leaves, and overall leaf area (cm2) alleles. For example, the markers of the present invention can be used in MAS in crosses involving elite and exotic Lettuce lines by subjecting the segregating progeny to MAS to maintain trait alleles. A breeder can then reliably track the inheritance of the trait alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having favorable trait alleles can be reliably predicted to express the desirable phenotype and progeny that share genotypes with the parent having unfavorable trait alleles can be reliably predicted to express the undesirable phenotype.

By providing the positions in the Lettuce genome of beneficial trait (plant (canopy) diameter (cm), plant height (cm), number of leaves, and overall leaf area (cm2))chromosome intervals and the associated markers within those intervals, the invention also allows one skilled in the art to identify and use other markers within the intervals disclosed herein or linked to the intervals disclosed herein. Having identified such regions, these markers can be readily identified from public linkage maps.

Closely linked markers flanking the locus of interest that have alleles in linkage disequilibrium with a trait allele at that locus may be effectively used to select for progeny plants with desirable trait. Thus, the markers described herein, such as those listed in Table 4, as well as other markers genetically linked to the same chromosome interval, may be used to select for Lettuce plants with plant (canopy) diameter (cm), plant height (cm), number of leaves, and overall leaf area (cm2). Often, a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

The choice of markers actually used to practice the invention is not limited and can be any marker that is genetically linked to the intervals as described herein, which includes markers mapping within the intervals. In certain embodiments, the invention further provides markers closely genetically linked to, or within approximately 0.5 cM of, the markers provided herein and chromosome intervals whose borders fall between or include such markers, and including markers within approximately 0.4 cM, 0.3 cM, 0.2 cM, and about 0.1 cM of the markers provided herein. Furthermore, since there are many different types of marker detection assays known in the art, it is not intended that the type of marker detection assay used to practice this invention be limited in any way.

### Molecular Markers

“Marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

“Marker” also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A favorable allele of a marker is the allele of the marker that co-segregates with a desired phenotype (e.g., plant (canopy) diameter (cm), plant height (cm), number of leaves, and overall leaf area (cm2)). As used herein, a QTL marker has a minimum of one favorable allele, although it is possible that the marker might have two or more favorable alleles found in the population. Any favorable allele of that marker can be used advantageously for the identification and construction of plant lines having the desired phenotype. Optionally, one, two, three or more favorable allele(s) of different markers are identified in, or introgressed into a plant, and can be selected for or against during MAS. Desirably, plants or germplasm are identified that have at least one such favorable allele that positively correlates with plant (canopy) diameter (cm), plant height (cm), number of leaves, and overall leaf area (cm2). Alternatively, a marker allele that co-segregates with trait also finds use with the invention, since that allele can be used to identify and counter select this trait in plants. Such an allele can be used for exclusionary purposes during breeding to identify alleles that negatively correlate with trait, to eliminate plants or germplasm having undesirable phenotypes from subsequent rounds of breeding.

The more tightly linked a marker is with a DNA locus influencing a phenotype, the more reliable the marker is in MAS, as the likelihood of a recombination event unlinking the marker and the locus decreases. Markers containing the causal mutation for a trait, or that are within the coding sequence of a causative gene, are ideal as no recombination is expected between them and the sequence of DNA responsible for the phenotype.

Genetic markers are distinguishable from each other (as well as from the plurality of alleles of any one particular marker) on the basis of polynucleotide length and/or sequence. In general, any differentially inherited polymorphic trait (including a nucleic acid polymorphism) that segregates among progeny is a potential genetic marker.

In some embodiments of the invention, one or more marker alleles are selected for in a single plant or a population of plants. In these methods, plants are selected that contain favorable alleles from more than one marker, or alternatively, favorable alleles from more than one marker are introgressed into a desired germplasm. One of skill recognizes that the identification of favorable marker alleles is germplasm-specific. The determination of which marker alleles correlate with plant (canopy) diameter (cm), plant height (cm), number of leaves, and overall leaf area (cm2) is determined for the particular germplasm under study. One of skill recognizes that methods for identifying the favorable alleles are routine and well known in the art, and furthermore, that the identification and use of such favorable alleles is well within the scope of this invention. Identification of favorable marker alleles in plant populations other than the populations used or described herein is well within the scope of this invention.

### Marker Detection

In some aspects, methods of the invention utilize an amplification step to detect/genotype a marker locus, but amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

“Amplifying,” in the context of nucleic acid amplification, is any process whereby additional copies of a selected nucleic acid (or a transcribed form thereof) are produced. In some embodiments, an amplification-based marker technology is used wherein a primer or amplification primer pair is admixed with genomic nucleic acid isolated from the first plant or germplasm, and wherein the primer or primer pair is complementary or partially complementary to at least a portion of the marker locus, and is capable of initiating DNA polymerization by a DNA polymerase using the plant genomic nucleic acid as a template. The primer or primer pair is extended in a DNA polymerization reaction having a DNA polymerase and a template genomic nucleic acid to generate at least one amplicon. In other embodiments, plant RNA is the template for the amplification reaction. In some embodiments, the QTL marker is a SNP type marker, and the detected allele is a SNP allele, and the method of detection is allele specific hybridization (ASH).

In general, the majority of genetic markers rely on one or more properties of nucleic acids for their detection. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like). A “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A “template nucleic acid” is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In one embodiment, the presence or absence of a molecular marker is determined simply through nucleotide sequencing of the polymorphic marker region. This method is readily adapted to high throughput analysis as are the other methods noted above, e.g., using available high throughput sequencing methods such as sequencing by hybridization.

In alternative embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST®, or even simple word processors.

While the exemplary markers provided in the tables herein are SNP markers, any of the aforementioned marker types can be employed in the context of the invention to identify chromosome intervals encompassing genetic element that contribute to plant (canopy) diameter (cm), plant height (cm), number of leaves, and overall leaf area (cm2).

### Probes and Primers

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the invention be limited to any particular size.

In some embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. As used in the art, PCR primers used to amplify a molecular marker are sometimes termed “PCR markers” or simply “markers.” It will be appreciated that, although many specific examples of primers are provided herein, suitable primers to be used with the invention can be designed using any suitable method. It is not intended that the invention be limited to any particular primer or primer pair. In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. Marker alleles in addition to those recited herein also find use with the present invention.

### Linkage Analysis

“Linkage”, or “genetic linkage,” is used to describe the degree with which one marker locus is associated with another marker locus or some other locus (for example, a plant (canopy) diameter (cm), plant height (cm), number of leaves, and overall leaf area (cm2) locus). A marker locus may be located within a locus to which it is genetically linked. For example, if locus A has genes “A” or “a” and locus B has genes “B” or “b” and a cross between parent 1 with AABB and parent 2 with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ is attributed to linkage. As used herein, linkage can be between two markers, or alternatively between a marker and a phenotype. A marker locus may be genetically linked to a trait, and in some cases a marker locus genetically linked to a trait is located within the allele conferring the trait. A marker may also be causative for a trait or phenotype, for example a causative polymorphism. The degree of linkage of a molecular marker to a phenotypic trait (e.g., a QTL) is measured, e.g., as a statistical probability of co-segregation of that molecular marker with the phenotype.

As used herein, “closely linked” means that the marker or locus is within about 20 cM, for instance within about 10 cM, about 5 cM, about 1 cM, about 0.5 cM, or less than 0.5 cM of the identified locus associated with plant (canopy) diameter (cm), plant height (cm), number of leaves, and overall leaf area (cm2).

As used herein, the linkage relationship between a molecular marker and a phenotype is given is the statistical likelihood that the particular combination of a phenotype and the presence or absence of a particular marker allele is random. Thus, the lower the probability score, the greater the likelihood that a phenotype and a particular marker will cosegregate. In some embodiments, a probability score of 0.05 (p=0.05, or a 5% probability) of random assortment is considered a significant indication of co-segregation. However, the present invention is not limited to this particular standard, and an acceptable probability can be any probability of less than 50% (p<0.5). For example, a significant probability can be less than 0.25, less than 0.20, less than 0.15, or less than 0.1. The phrase “closely linked,” in the present application, means that recombination between two linked loci occurs with a frequency of equal to or less than about 10% (i.e., are separated on a genetic map by not more than 10 cM). In one aspect, any marker of the invention is linked (genetically and physically) to any other marker that is at or less than 50 cM distant. In another aspect, any marker of the invention is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

Classical linkage analysis can be thought of as a statistical description of the relative frequencies of cosegregation of different traits. Linkage analysis is the well characterized descriptive framework of how traits are grouped together based upon the frequency with which they segregate together. That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. The further apart on a chromosome the genes reside, the less likely they are to segregate together, because homologous chromosomes recombine during meiosis. Thus, the further apart on a chromosome the genes reside, the more likely it is that there will be a crossing over event during meiosis that will result in the marker and the DNA sequence responsible for the trait the marker is designed to track segregating separately into progeny. A common measure of linkage is the frequency with which traits cosegregate.

Linkage analysis is used to determine which polymorphic marker allele demonstrates a statistical likelihood of co-segregation with a desired trait phenotype (a “trait marker allele”). Following identification of a marker allele for co-segregation with the trait phenotype, it is possible to use this marker for rapid, accurate screening of plant lines for plant (canopy) diameter (cm), plant height (cm), number of leaves, and overall leaf area (cm2) alleles without the need to grow the plants through their life cycle and await phenotypic evaluations, and furthermore, permits genetic selection for the particular allele even when the molecular identity of the actual trait QTL is unknown. Tissue samples can be taken, for example, from the endosperm, embryo, or mature/developing plant and screened with the appropriate molecular marker to rapidly determine determined which progeny contain the desired genetics. Linked markers also remove the impact of environmental factors that can often influence phenotypic expression.

Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, in the context of the present invention, one cM is equal to a 1% chance that a marker locus will be separated from another locus (which can be any other trait, e.g., another marker locus, or another trait locus that encodes a QTL), due to crossing over in a single generation.

When referring to the relationship between two genetic elements, such as a genetic element contributing to trait and a proximal marker, “coupling” phase linkage indicates the state where the “favorable” allele at the plant (canopy) diameter (cm), plant height (cm), number of leaves, and overall leaf area (cm2) locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for trait) is physically linked with an “unfavorable” allele at the proximal marker locus, and the two “favorable” alleles are not inherited together (i.e., the two loci are “out of phase” with each other).

### Genetic Mapping

A “genetic map” is the relationship of genetic linkage among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map. A “genetic recombination frequency” is the frequency of a crossing over event (recombination) between two genetic loci. Recombination frequency can be observed by following the segregation of markers and/or traits following meiosis. In some cases, two different markers can have the same genetic map coordinates. In that case, the two markers are in such close proximity to each other that recombination occurs between them with such low frequency that it is undetected.

Genetic maps are graphical representations of genomes (or a portion of a genome such as a single chromosome) where the distances between markers are measured by the recombination frequencies between them. Plant breeders use genetic maps of molecular markers to increase breeding efficiency through MAS, a process where selection for a trait of interest is not based on the trait itself but rather on the genotype of a marker linked to the trait. A molecular marker that demonstrates reliable linkage with a phenotypic trait provides a useful tool for indirectly selecting the trait in a plant population, especially when accurate phenotyping is difficult, slow, or expensive.

In general, the closer two markers or genomic loci are on the genetic map, the closer they lie to one another on the physical map. A lack of precise proportionality between cM distances and physical distances can exist due to the fact that the likelihood of genetic recombination is not uniform throughout the genome; some chromosome regions are cross-over “hot spots,” while other regions demonstrate only rare recombination events, if any.

Genetic mapping variability can also be observed between different populations of the same crop species. In spite of this variability in the genetic map that may occur between populations, genetic map and marker information derived from one population generally remains useful across multiple populations in identification of plants with desired traits, counter-selection of plants with undesirable traits and in guiding MAS.

As one of skill in the art will recognize, recombination frequencies (and as a result, genetic map positions) in any particular population are not static. The genetic distances separating two markers (or a marker and a QTL) can vary depending on how the map positions are determined. For example, variables such as the parental mapping populations used, the software used in the marker mapping or QTL mapping, and the parameters input by the user of the mapping software can contribute to the QTL marker genetic map relationships. However, it is not intended that the invention be limited to any particular mapping populations, use of any particular software, or any particular set of software parameters to determine linkage of a particular marker or chromosome interval with a desired phenotype. It is well within the ability of one of ordinary skill in the art to extrapolate the novel features described herein to any gene pool or population of interest, and using any particular software and software parameters. Indeed, observations regarding genetic markers and chromosome intervals in populations in addition to those described herein are readily made using the teaching of the present disclosure.

### Association Mapping

Association or LD mapping techniques aim to identify genotype-phenotype associations that are significant. It is effective for fine mapping in outcrossing species where frequent recombination among heterozygotes can result in rapid LD decay. LD is non-random association of alleles in a collection of individuals, reflecting the recombinational history of that region. Thus, LD decay averages can help determine the number of markers necessary for a genome-wide association study to generate a genetic map with a desired level of resolution.

Large populations are better for detecting recombination, while older populations are generally associated with higher levels of polymorphism, both of which contribute to accelerated LD decay. However, smaller effective population sizes tend to show slower LD decay, which can result in more extensive haplotype conservation. Understanding of the relationships between polymorphism and recombination is useful in developing strategies for efficiently extracting information from these resources. Association analyses compare the plants' phenotypic score with the genotypes at the various loci. Subsequently, any suitable maize genetic map (for example, a composite map) can be used to help observe distribution of the identified QTL markers and/or QTL marker clustering using previously determined map locations of the markers.

### Marker Assisted Selection

“Introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A primary motivation for development of molecular markers in crop species is the potential for increased efficiency in plant breeding through MAS. Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present invention provides the means to identify plants that exhibit plant (canopy) diameter (cm), plant height (cm), number of leaves, and overall leaf area (cm2) by identifying plants having a specified allele that is linked to the trait locus on chromosomes identified herein.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneous selected and/or introgressed. The combinations of markers that are selected for in a single plant is not limited, and can include any combination of markers disclosed herein or any marker linked to the markers disclosed herein, or any markers located within the QTL intervals defined herein.

In some embodiments, a first Lettuce plant or germplasm exhibiting a desired trait (the donor) can be crossed with a second Lettuce plant or germplasm (the recipient, e.g., an elite or exotic Lettuce, depending on characteristics that are desired in the progeny) to create an introgressed Lettuce plant or germplasm as part of a breeding program. In some aspects, the recipient plant can also contain one or more loci associated with one or more desired traits, which can be qualitative or quantitative trait loci. In another aspect, the recipient plant can contain a transgene.

In some embodiments, the recipient Lettuce plant or germplasm will typically display less desirable trait as compared to the first Lettuce plant or germplasm, while the introgressed Lettuce plant or germplasm will exhibit plant (canopy) diameter (cm), plant height (cm), number of leaves, and overall leaf area (cm2) as compared to the second plant or germplasm. An introgressed Lettuce plant or germplasm produced by these methods are also a feature of this invention.

MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

When a population is segregating for multiple loci affecting one of multiple traits, e.g., multiple loci involved in trait, the efficiency of MAS compared to phenotypic screening becomes even greater, because all of the loci can be evaluated in the lab together from a single sample of DNA.

### Introgression of Trait Loci Using MAS

The introgression of one or more desired loci from a donor line into another is achieved via repeated backcrossing to a recurrent parent accompanied by selection to retain one or more loci from the donor parent. Markers associated with plant (canopy) diameter (cm), plant height (cm), number of leaves, and overall leaf area (cm2) are assayed in progeny and those progeny with one or more desired markers are selected for advancement. In another aspect, one or more markers can be assayed in the progeny to select for plants with the genotype of the elite parent. This invention anticipates that trait introgression activities will require more than one generation, wherein progeny are crossed to the recurrent (elite) parent or selfed. Selections are made based on the presence of one or more plant (canopy) diameter (cm), plant height (cm), number of leaves, and overall leaf area (cm2) markers and can also be made based on the recurrent parent genotype, wherein screening is performed on a genetic marker and/or phenotype basis. In another embodiment, markers of this invention can be used in conjunction with other markers, ideally at least one on each chromosome of the Lettuce genome, to track the introgression of plant (canopy) diameter (cm), plant height (cm), number of leaves, and overall leaf area (cm2) loci into elite germplasm. It is within the scope of this invention to utilize the methods and compositions for trait integration of plant (canopy) diameter (cm), plant height (cm), number of leaves, and overall leaf area (cm2). It is contemplated by the inventors that the present invention will be useful for developing commercial varieties with plant (canopy) diameter (cm), plant height (cm), number of leaves, and overall leaf area (cm2) and an elite phenotype.

In one aspect, this invention could be used on any plant. In another aspect, the plant is selected from the genus Lettuca. In another aspect, the plant is selected from the species Lettuca sativa.

In another aspect, a Lettuce plants from the cultivars of the invention can show a unique combination of traits including canopy size number of leaves and the like compared to a traditional Lettuce plant. In this aspect, a traditional Lettuce variety includes ones that are wildtype and commercially grown.

### Further Embodiments of the Invention

This invention is also directed to methods for producing a Lettuce plant by crossing a first parent Lettuce plant with a second parent Lettuce plant, wherein the first parent Lettuce plant or second parent Lettuce plant is the Lettuce plant from cultivar VINDARA_13, VINDARA_16, and VINDARA_18. Further, both the first parent Lettuce plant and second parent Lettuce plant may be from cultivar VINDARA_13, VINDARA_16, and VINDARA_18. Therefore, any methods using Lettuce cultivar VINDARA_13, VINDARA_16, and VINDARA_18 are part of this invention, such as selfing, backcrosses, hybrid breeding, and crosses to populations. Plants produced using Lettuce cultivar VINDARA_13, VINDARA_16, and VINDARA_18 as at least one parent are within the scope of this invention.

In one aspect of the invention, methods for developing novel plant types are presented. In one embodiment the specific type of breeding method is pedigree selection, where both single plant selection and mass selection practices are employed. Pedigree selection, also known as the “Vilmorin system of selection,” is described in Fehr, Walter; Principles of Cultivar Development, Volume I, Macmillan Publishing Co., which is hereby incorporated by reference.

In one embodiment, the pedigree method of breeding is practiced where selection is first practiced among F₂ plants. In the next season, the most desirable F₃ lines are first identified, and then desirable F₃ plants within each line are selected. The following season and in all subsequent generations of inbreeding, the most desirable families are identified first, then desirable lines within the selected families are chosen, and finally desirable plants within selected lines are harvested individually. A family refers to lines that were derived from plants selected from the same progeny row the preceding generation.

Using this pedigree method, two parents may be crossed using an emasculated female and a pollen donor (male) to produce F₁ offspring. The F₁ may be self-pollinated to produce a segregating F₂ generation. Individual plants may then be selected which represent the desired phenotype in each generation (F₃, F₄, F₅, etc.) until the traits are homozygous or fixed within a breeding population.

In addition to crossing, selection may be used to identify and isolate new Lettuce lines. In Lettuce selection, Lettuce seeds are planted, the plants are grown and single plant selections are made of plants with desired characteristics. Seed from the single plant selections may be harvested, separated from seeds of the other plants in the field and re-planted. The plants from the selected seed may be monitored to determine if they exhibit the desired characteristics of the originally selected line. Selection work is preferably continued over multiple generations to increase the uniformity of the new line.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives but should include gain from selection per year based on comparisons to an appropriate standard, the overall value of the advanced breeding lines, and the number of successful cultivars produced per unit of input (e.g., per year, per dollar expended, etc.).

In one embodiment, promising advanced breeding lines are thoroughly tested and compared to appropriate standards in environments representative of the commercial target area(s). The best lines are candidates for new commercial cultivars; those still deficient in a few traits are used as parents to produce new populations for further selection.

These processes, which lead to the final step of marketing and distribution, usually take several years from the time the first cross or selection is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of Lettuce plant breeding is to develop new, unique and superior Lettuce cultivars. In one embodiment, the breeder initially selects and crosses two or more parental lines, followed by repeated selfing and selection, producing many new genetic combinations. The breeder can theoretically generate billions of different genetic combinations via crossing, selfing and mutations. Preferably, each year the plant breeder selects the germplasm to advance to the next generation. This germplasm may be grown under different geographical, climatic and soil conditions, and further selections are then made, during and at the end of the growing season.

In a preferred embodiment, the development of commercial Lettuce cultivars requires the development of Lettuce varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Pedigree breeding is used commonly for the improvement of self-pollinating crops or inbred lines of cross-pollinating crops. Two parents which possess favorable, complementary traits are crossed to produce an F₁. An F₂ population is produced by selfing one or several F₁'s or by intercrossing two F₁'s (sib mating). Selection of the best individuals is usually begun in the F₂ population; then, beginning in the F₃, the best individuals in the best families are usually selected. Replicated testing of families, or hybrid combinations involving individuals of these families, often follows in the F₄ generation to improve the effectiveness of selection for traits with low heritability. At an advanced stage of inbreeding (e.g., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

The single-seed descent procedure refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the identification of markers which are closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into Lettuce varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Principles of Plant Breeding John Wiley and Son, pp. 115-161, 1960; Allard, 1960; Simmonds, 1979; Sneep et al., 1979; Fehr, 1987; “Carrots and Related Vegetable Umbelliferae”, Rubatzky, V. E., et al., 1999).

Lettuce is an important and valuable crop. Thus, a continuing goal of Lettuce plant breeders is to develop stable, high yielding Lettuce cultivars that are agronomically sound. To accomplish this goal, the Lettuce breeder preferably selects and develops Lettuce plants with traits that result in superior cultivars.

This invention also is directed to methods for producing a Lettuce cultivar plant by crossing a first parent Lettuce plant with a second parent Lettuce plant wherein either the first or second parent Lettuce plant is a Lettuce plant of the line VINDARA_13, VINDARA_16, and/or VINDARA_18. Further, both first and second parent Lettuce plants can come from the cultivar VINDARA_13, VINDARA_16, and/or VINDARA_18. Still further, this invention also is directed to methods for producing a cultivar VINDARA_13, VINDARA_16, and/or VINDARA_18-derived Lettuce plant by crossing cultivar VINDARA_13, VINDARA_16, and/or VINDARA_18 with a second Lettuce plant and growing the progeny seed, and repeating the crossing and growing steps with the cultivar VINDARA_13, VINDARA_16, and/or VINDARA_18-derived plant from 0 to 7 times. Thus, any such methods using the cultivar VINDARA_13, VINDARA_16, and/or VINDARA_18 are part of this invention: selfing, backcrosses, hybrid production, crosses to populations, and the like. All plants produced using cultivar VINDARA_13, VINDARA_16, and/or VINDARA_18 as a parent are within the scope of this invention, including plants derived from cultivar VINDARA_13, VINDARA_16, and/or VINDARA_18. Advantageously, the cultivar is used in crosses with other, different, cultivars to produce first generation (F₁) Lettuce seeds and plants with superior characteristics.

As used herein, the term plant includes plant cells, plant protoplasts, plant cell tissue cultures from which Lettuce plants can be regenerated, plant calli, plant clumps and plant cells that are intact in plants or parts of plants, such as embryos, pollen, ovules, flowers, seeds, roots, anthers, and the like.

As is well known in the art, tissue culture of Lettuce can be used for the in vitro regeneration of a Lettuce plant. Tissue culture of various tissues of Lettuce and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672. It is clear from the literature that the state of the art is such that these methods of obtaining plants are, and were, “conventional” in the sense that they are routinely used and have a very high rate of success. Thus, another aspect of this invention is to provide cells which upon growth and differentiation produce Lettuce plants having the physiological and morphological characteristics of variety VINDARA_13, VINDARA_16, and/or VINDARA_18.

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as transgenes. Over the last fifteen to twenty years several methods for producing transgenic plants have been developed, and the present invention, in particular embodiments, also relates to transformed versions of the claimed line.

Plant transformation preferably involves the construction of an expression vector that will function in plant cells. Such a vector may comprise DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed Lettuce plants, using transformation methods as described below to incorporate transgenes into the genetic material of the Lettuce plant(s).

### Expression Vectors for Lettuce Transformation

### Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, aminoglycoside-3′-adenyl transferase, the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990<Hille et al., Plant Mol. Biol. 7:171 (1986). Other selectable marker genes confer resistance to herbicides such as glyphosate, glufosinate or broxynil. Comai et al., Nature 317:741-744 (1985), Gordon-Kamm et al., Plant Cell 2:603-618 (1990) and Stalker et al., Science 242:419-423 (1988).

Other selectable marker genes for plant transformation are not of bacterial origin. These genes include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta.-glucuronidase (GUS), .beta.-galactosidase, luciferase and chloramphenicol, acetyltransferase. Jefferson, R. A., Plant Mol. Biol. Rep. 5:387 (1987), Teen et al., EMBO J. 8:343 (1989), Koncz et al., Proc. Natl. Acad. Sci U.S.A. 84:131 (1987), DeBlock et al., EMBO J. 3:1681 (1984).

Recently, in vivo methods for visualizing GUS activity that do not require destruction of plant tissue have been made available. Molecular Probes publication 2908, Imagene Green™, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Promoters

Genes included in expression vectors preferably are driven by nucleotide sequence comprising a regulatory element, for example, a promoter. Several types of promoters are now well known in the transformation arts, as are other regulatory elements that can be used alone or in combination with promoters.

As used herein, promoter includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive promoter” is a promoter which is active under most environmental conditions.

A. Inducible Promoters

An inducible promoter is operably linked to a gene for expression in Lettuce. Optionally, the inducible promoter is operably linked to a nucleotide sequence encoding a signal sequence which is operably linked to a gene for expression in Lettuce. With an inducible promoter the rate of transcription increases in response to an inducing agent. Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. U.S.A. 88:0421 (1991).

B. Constitutive Promoters

A constitutive promoter may be operably linked to a gene for expression in Lettuce or the constitutive promoter may operably linked to a nucleotide sequence encoding a signal sequence which is operably linked to a gene for expression in Lettuce.

Many different constitutive promoters can be utilized in the instant invention. Exemplary constitutive promoters include, but are not limited to, the promoters from plant viruses such as the 35S promoter from CaMV (Odell et al., Nature 313:810-812 (1985) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO96/30530.

C. Tissue-Specific or Tissue Preferred Promoters

A tissue-specific promoter may be operably linked to a gene for expression in Lettuce. Optionally, the tissue-specific promoter is operably linked to a nucleotide sequence encoding a signal sequence which is operably linked to a gene for expression in Lettuce. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993).

### Signal Sequences for Targeting Proteins to Subcellular Compartments

Transport of protein produced by transgenes to a subcellular compartment such as the chloroplast, vacuole, peroxisome, glyoxysome, cell wall or mitochondroin or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992), Close, P. S., Master's Thesis, Iowa State University (1993), Knox, C., et al., Structure and Organization of Two Divergent Alpha-Amylase Genes from Barley, Plant Mol. Biol. 9:3-17 (1987), Lerner et al., Plant Physiol. 91:124-129 (1989), Fontes et al., Plant Cell 3:483-496 (1991), Matsuoka et al., Proc. Natl. Acad. Sci. 88:834 (1991), Gould et al., J. Cell. Biol. 108:1657 (1989), Creissen et al., Plant J. 2:129 (1991), Kalderon, et al., A short amino acid sequence able to specify nuclear location, Cell 39:499-509 (1984), Steifel, et al., Expression of a maize cell wall hydroxyproline-rich glycoprotein gene in early leaf and root vascular differentiation, Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants that are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is Lettuce. In another preferred embodiment, the biomass of interest is seed. For transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons may involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant line can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487, the contents of which are hereby incorporated by reference. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotoch. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper accumulation of a monterpene, a sesquiterpene, a steroid, hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hookworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung Lettuce calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of tachyolesin which inhibit fungal plant pathogens) and PCT application WO95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-β, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb at al., Bio/Technology 10:1436 (1992). The cloning and characterization of a gene which encodes a Lettuce endopolygalacturonase-inhibiting protein is described by Toubart et al., Plant J. 2:367 (1992).

R. A development-arrestive protein produced in nature by a plant. For example, Logemann et al., Bioi/Technology 10:305 (1992), have shown that transgenic plants expressing the barley ribosome-inactivating gene have an increased resistance to fungal disease.

S. A Lettuce mosaic potyvirus (LMV) coat protein gene introduced into Lactuca sativa in order to increase its resistance to LMV infection. See Dinant et al., Molecular Breeding. 1997, 3: 1, 75-86.

2. Genes that Confer Resistance to an Herbicide, for Example:

A. A herbicide that inhibits the growing point or meristem, such as an imidazalinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance impaired by mutant 5-enolpyruvl-3-phosphikimate synthase (EPSP) and aroA genes, respectively) and other phosphono compounds such as glufosinate (phosphinothricin acetyl transferase, PAT and Streptomyces hygroscopicus phosphinothricin-acetyl transferase PAT bar genes), and pyridinoxy or phenoxy propionic acids and cycloshexones (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. See also Umaballava-Mobapathie in Transgenic Research. 1999, 8: 1, 33-44 that discloses Lactuca sativa resistant to glufosinate. European patent application No. 0 333 033 to Kumada at al., and U.S. Pat. No. 4,975,374 to Goodman et al., disclose nucleotide sequences of glutamine synthetase genes which confer resistance to herbicides such as L-phosphinothricin. The nucleotide sequence of a phosphinothricin-acetyl-transferase gene is provided in European application No. 0 242 246 to Leemans et al., DeGreef et al., Bio/Technology 7:61 (1989), describe the production of transgenic plants that express chimeric bar genes coding for phosphinothricin acetyl transferase activity. Exemplary of genes conferring resistance to phenoxy propionic acids and cycloshexones, such as sethoxydim and haloxyfop are the Acc1-S1, Acc1-S2 and Acc1-S3 genes described by Marshall et al., Theor. Appl. Genet. 83:435 (1992).

C. A herbicide that inhibits photosynthesis, such as a triazine (psbA and gs+ genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and international publication WO 01/12825.

3. Genes that Confer or Contribute to a Value-Added Trait, Such as:

A. Increased iron content of the Lettuce, for example by transforming a plant with a soybean ferritin gene as described in Goto et al., Acta Horticulturae. 2000, 521, 101-109. Parallel to the improved iron content enhanced growth of transgenic Lettuce s was also observed in early development stages.

B. Decreased nitrate content of leaves, for example by transforming a Lettuce with a gene coding for a nitrate reductase. See for example Curtis et al., Plant Cell Report. 1999, 18: 11, 889-896.

C. Increased sweetness of the Lettuce by transferring a gene coding for monellin that elicits a flavor sweeter than sugar on a molar basis. See Penarrubia et al., Biotechnology. 1992, 10: 5, 561-564.

D. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

E. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus licheniformis α-amylase), Elliot et al., Plant Molec. Biol. 21:515 (1993) (nucleotide sequences of tomato invertase genes), Sogaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

4. Genes that Control Male-Sterility

A. Introduction of a deacetylase gene under the control of a tapetum-specific promoter and with the application of the chemical N—Ac-PPT. See international publication WO 01/29237.

B. Introduction of various stamen-specific promoters. See international publications WO 92/13956 and WO 92/13957.

C. Introduction of the barnase and the barstar genes. See Paul et al., Plant Mol. Biol. 19:611-622, 1992).

### Methods for Lettuce Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-Mediated Transformation

One method for introducing an expression vector into plants is based on the natural transformation system of Agrobacterium. See, for example, Horsch et al., Science 227:1229 (1985). Curtis et al., Journal of Experimental Botany. 1994, 45: 279, 1441-1449, Tones et al., Plant cell Tissue and Organic Culture. 1993, 34: 3, 279-285, Dinant et al., Molecular Breeding. 1997, 3: 1, 75-86. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer

Several methods of plant transformation collectively referred to as direct gene transfer have been developed as an alternative to Agrobacterium-mediated transformation. A generally applicable method of plant transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Russell, D. R., et al. Pl. Cell. Rep. 12(3, January), 165-169 (1993), Aragao, F. J. L., et al. Plant Mol. Biol. 20(2, October), 357-359 (1992), Aragao, F. J. L., et al. Pl. Cell. Rep. 12(9, July), 483-490 (1993). Aragao, Theor. Appl. Genet. 93: 142-150 (1996), Kim, J.; Minamikawa, T. Plant Science 117: 131-138 (1996), Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion has been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂) precipitation, polyvinyl alcohol or poly-L-omithine has also been reported. Hain et al., Mol. Gen. Genet. 199:161 (1985) and Draper et al., Plant Cell Physiol. 23:451 (1982). Electroporation of protoplasts and whole cells and tissues have also been described. Saker, M.; Kuhne, T. Biologia Plantarum 40(4): 507-514 (1997/98), Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994). See also Chupean et al., Biotechnology. 1989, 7: 5, 503-508.

Following transformation of Lettuce target tissues, expression of the above-described selectable marker genes allows for preferential selection of transformed cells, tissues and/or plants, using regeneration and selection methods now well known in the art.

The foregoing methods for transformation would typically be used for producing a transgenic line. The transgenic line could then be crossed, with another (non-transformed or transformed) line, in order to produce a new transgenic Lettuce line. Alternatively, a genetic trait that has been engineered into a particular Lettuce cultivar using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene. As used herein, “crossing” can refer to a simple X by Y cross, or the process of backcrossing, depending on the context.

### Sequence Identity

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. GenBank® is the recognized United States-NIH genetic sequence database, comprising an annotated collection of publicly available DNA sequences, and which further incorporates submissions from the European Molecular Biology Laboratory (EMBL) and the DNA DataBank of Japan (DDBJ), see Nucleic Acids Research, January 2013, v 41(D1) D36-42 for discussion. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein, for instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9), or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

### Tilling

In one embodiment, TILLING (Targeting Induced Local Lesions IN Genomes) can be used to produce plants in which endogenous genes comprise a mutation, for example genes increase plant (canopy) diameter (cm), plant height (cm), number of leaves, and overall leaf area (cm2). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time. For a TILLING assay, heteroduplex methods using specific endonucleases can be used to detect single nucleotide polymorphisms (SNPs). Alternatively, Next Generation Sequencing of DNA from pools of mutagenized plants can be used to identify mutants in the gene of choice. Typically, a mutation frequency of one mutant per 1000 plants in the mutagenized population is achieved. Using this approach, many thousands of plants can be screened to identify any individual with a single base change as well as small insertions or deletions (1-30 bp) in any gene or specific region of the genome. TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

### Genome Editing Using Site-Specific Nucleases

Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption. Engineered nucleases useful in the methods of the present invention include zinc finger nucleases (ZFNs), transcription activator-like (TAL) effector nucleases (TALEN) and CRISPR/Cas9 type nucleases.

Typically, nuclease encoded genes are delivered into cells by plasmid DNA, viral vectors or in vitro transcribed mRNA. A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis2His2 type of zinc finger that is very generally represented, for example, by the zinc finger transcription factors TFIIIA or Sp1. In a preferred embodiment, the zinc finger domain comprises three Cis2His2 type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques (see, for example, Bibikova et al., 2002).

The ZFN DNA-cleavage domain is derived from a class of non-specific DNA cleavage domains, for example the DNA-cleavage domain of a Type II restriction enzyme such as Fold (Kim et al., 1996). Other useful endonucleases may include, for example, HhaI, HindIII, Nod, BbvCI, EcoRI, BglI, and AlwI.

A transcription activator-like (TAL) effector nuclease (TALEN) comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences. Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations, via RNA-guided DNA cleavage.

CRISPR systems rely on CRISPR RNA (crRNA) and transactivating chimeric RNA (tracrRNA) for sequence-specific cleavage of DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components. The Cas endonuclease may be converted into a nickase to provide additional control over the mechanism of DNA repair (Cong et al., 2013). CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

### Gene Conversions

When the term Lettuce plant, cultivar or Lettuce line is used in the context of the present invention, this also includes any gene conversions of that line. The term gene converted plant as used herein refers to those Lettuce plants which are developed by a plant breeding technique called backcrossing wherein essentially all of the desired morphological and physiological characteristics of a cultivar are recovered in addition to the gene transferred into the line via the backcrossing technique. Backcrossing methods can be used with the present invention to improve or introduce a characteristic into the line. The term backcrossing as used herein refers to the repeated crossing of a hybrid progeny back to one of the parental Lettuce plants for that line. The parental Lettuce plant that contributes the gene for the desired characteristic is termed the nonrecurrent or donor parent. This terminology refers to the fact that the nonrecurrent parent is used one time in the backcross protocol and therefore does not recur. The parental Lettuce plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a Lettuce plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute traits or characteristics in the original line. To accomplish this, a gene or genes of the recurrent cultivar are modified or substituted with the desired gene or genes from the nonrecurrent parent, while retaining essentially all of the rest of the desired genetic, and therefore the desired physiological and morphological, constitution of the original line. The choice of the particular nonrecurrent parent will depend on the purpose of the backcross. One of the major purposes is to add some commercially desirable, agronomically important trait or traits to the plant. The exact backcrossing protocol will depend on the characteristics or traits being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many gene traits have been identified that are not regularly selected for in the development of a new line but that can be improved by backcrossing techniques. Gene traits may or may not be transgenic, examples of these traits include but are not limited to, herbicide resistance, resistance for bacterial, fungal, or viral disease, insect resistance, enhanced nutritional quality, industrial usage, yield stability, yield enhancement, male sterility, modified fatty acid metabolism, and modified carbohydrate metabolism. These genes are generally inherited through the nucleus. Several of these gene traits are described in U.S. Pat. Nos. 5,777,196; 5,948,957 and 5,969,212, the disclosures of which are specifically hereby incorporated by reference.

### Tissue Culture

Further reproduction of the variety can occur by tissue culture and regeneration. Tissue culture of various tissues of Lettuce and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672, and Ibrahim et al., Plant Cell, Tissue and Organ Culture. (1992), 28(2): 139-145. It is clear from the literature that the state of the art is such that these methods of obtaining plants are routinely used and have a very high rate of success. Thus, another aspect of this invention is to provide cells which upon growth and differentiation produce Lettuce plants having the physiological and morphological characteristics of cultivar VINDARA_13, VINDARA_16, and/or VINDARA_18.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Additional Breeding Methods

This invention also is directed to methods for producing a Lettuce plant by crossing a first parent Lettuce plant with a second parent Lettuce plant wherein the first or second parent Lettuce plant is a Lettuce plant of cultivar VINDARA_13, VINDARA_16, and/or VINDARA_18. Further, both first and second parent Lettuce plants can come from Lettuce cultivar VINDARA_13, VINDARA_16, and/or VINDARA_18. Thus, any such methods using Lettuce cultivar VINDARA_13, VINDARA_16, and/or VINDARA_18 are part of this invention: selfing, backcrosses, hybrid production, crosses to populations, and the like. All plants produced using Lettuce cultivar VINDARA_13, VINDARA_16, and/or VINDARA_18 as at least one parent are within the scope of this invention, including those developed from cultivars derived from Lettuce cultivar VINDARA_13, VINDARA_16, and/or VINDARA_18. Advantageously, this Lettuce cultivar could be used in crosses with other, different, Lettuce plants to produce the first generation (F₁) Lettuce hybrid seeds and plants with superior characteristics. The cultivar of the invention can also be used for transformation where exogenous genes are introduced and expressed by the cultivar of the invention. Genetic variants created either through traditional breeding methods using Lettuce cultivar VINDARA_13, VINDARA_16, and/or VINDARA_18 or through transformation of cultivar VINDARA_13, VINDARA_16, and/or VINDARA_18 by any of a number of protocols known to those of skill in the art are intended to be within the scope of this invention.

The following describes breeding methods that may be used with the Lettuce cultivar of the invention in the development of further Lettuce plants. One such embodiment is a method for developing cultivar VINDARA_13, VINDARA_16, and/or VINDARA_18 progeny Lettuce plants in a Lettuce plant breeding program comprising: obtaining the Lettuce plant, or a part thereof, of cultivar VINDARA_13, VINDARA_16, and/or VINDARA_18, utilizing said plant or plant part as a source of breeding material, and selecting a Lettuce cultivar of the invention progeny plant with molecular markers in common with cultivar VINDARA_13, VINDARA_16, and/or VINDARA_18 and/or with morphological and/or physiological characteristics selected from the characteristics listed in Table 1. Breeding steps that may be used in the Lettuce plant breeding program include pedigree breeding, backcrossing, mutation breeding, and recurrent selection. In conjunction with these steps, techniques such as RFLP-enhanced selection, genetic marker enhanced selection (for example SSR markers) and the making of double haploids may be utilized.

Another method which may be used involves producing a population of Lettuce cultivar VINDARA_13, VINDARA_16, and/or VINDARA_18-progeny Lettuce plants, comprising crossing cultivar VINDARA_13, VINDARA_16, and/or VINDARA_18 with another Lettuce plant, thereby producing a population of Lettuce plants, which, on average, derive 50% of their alleles from Lettuce cultivar VINDARA_13, VINDARA_16, and/or VINDARA_18. A plant of this population may be selected and repeatedly selfed or sibbed with a Lettuce cultivar resulting from these successive filial generations. One embodiment of this invention is the Lettuce cultivar produced by this method and that has obtained at least 50% of its alleles from Lettuce cultivar VINDARA_13, VINDARA_16, and/or VINDARA_18.

One of ordinary skill in the art of plant breeding would know how to evaluate the traits of two plant varieties to determine if there is no significant difference between the two traits expressed by those varieties. For example, see Fehr and Walt, Principles of Cultivar Development, p 261-286 (1987). Thus the invention includes Lettuce cultivar VINDARA_13, VINDARA_16, and/or VINDARA_18 progeny Lettuce plants comprising a combination of at least two cultivar VINDARA_13, VINDARA_16, and/or VINDARA_18 traits selected from the group consisting of those listed in Table 1 or the cultivar VINDARA_13, VINDARA_16, and/or VINDARA_18 combination of traits listed above, so that said progeny Lettuce plant is not significantly different for said traits than Lettuce cultivar VINDARA_13, VINDARA_16, and/or VINDARA_18 as determined at the 5% significance level when grown in the same environmental conditions. Using techniques described herein, molecular markers may be used to identify said progeny plant as a Lettuce cultivar VINDARA_13, VINDARA_16, and/or VINDARA_18 progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of Lettuce cultivar VINDARA_13, VINDARA_16, and/or VINDARA_18 may also be characterized through their filial relationship with Lettuce cultivar VINDARA 13, VINDARA_16, and/or VINDARA_18, as for example, being within a certain number of breeding crosses of Lettuce cultivar VINDARA_13, VINDARA_16, and/or VINDARA_18. A breeding cross is a cross made to introduce new genetics into the progeny, and is distinguished from a cross, such as a self or a sib cross, made to select among existing genetic alleles. The lower the number of breeding crosses in the pedigree, the closer the relationship between Lettuce cultivar VINDARA_13, VINDARA_16, and/or VINDARA_18 and its progeny. For example, progeny produced by the methods described herein may be within 1, 2, 3, 4 or 5 breeding crosses of Lettuce cultivar VINDARA_13, VINDARA_16, and/or VINDARA_18.

The foregoing invention has been described in detail by way of illustration and example for purposes of clarity and understanding. However, it will be obvious that certain changes and modifications such as single gene modifications and mutations, somaclonal variants, variant individuals selected from large populations of the plants of the instant variety and the like may be practiced within the scope of the invention, as limited only by the scope of the appended claims.

### Embodiments of the Invention

Applicant reserves the right to make the following.

1. A Lettuce cultivar with superior plant (canopy) diameter (cm), plant height (cm), number of leaves, and overall leaf area (cm²) as compared to traditional lettuce cultivars and which includes one or more of SEQ ID NO: 1-12.
2. A plant of the lettuce cultivar of claim 1.
3. The Lettuce cultivar of claim 1, wherein the cultivar has the cultivar of VINDARA_13, VINDARA_16, and/or VINDARA_18 as an ancestor.
4. A Lettuce cultivar designated VINDARA_13, VINDARA_16, and/or VINDARA_18, wherein a representative sample of seed of said cultivar was deposited under Accession No. PTA-______, PTA-______ or PTA-______.
5. Seed of Lettuce cultivar designated VINDARA_13, VINDARA_16, and/or VINDARA_18, wherein a representative sample of seed of said cultivar was deposited under Accession No. PTA-______, PTA-______ or PTA-______.
6. A Lettuce plant, or a part thereof, produced by growing the seed of claim 6.
7. A tissue culture of cells produced from the plant of claim 7, wherein said cells of the tissue culture are produced from a plant part selected from the group consisting of embryo, meristematic cell, leaf, cotyledon, hypocotyl, stem, root, root tip, pistil, anther, flower, seed and pollen.
8. A protoplast produced from the plant of claim 7.
9. A protoplast produced from the tissue culture of claim 8.
10. A Lettuce plant regenerated from the tissue culture of claim 10, wherein the plant has all of the morphological and physiological characteristics of cultivar VINDARA_13, VINDARA_16, and/or VINDARA_18, wherein a representative sample of seed was deposited under Accession No. PTA-______, PTA-______ or PTA-______.
11. A method for producing a hybrid Lettuce seed, wherein the method comprises: crossing the Lettuce plant of any of claims 1 to 5 with a different Lettuce plant and harvesting the resultant F₁ hybrid Lettuce seed.
12. A hybrid Lettuce seed produced by the method of claim 11.
13. A hybrid Lettuce plant, or a part thereof, produced by growing said hybrid seed of claim 11.
14. A method of producing a Lettuce plant derived from the Lettuce cultivar VINDARA_13, VINDARA_16, and/or VINDARA_18 wherein the method comprises:
(a) crossing the plant of claim 7 with a second Lettuce plant to produce a progeny plant;
(b) crossing the progeny plant of step (a) with itself or the second Lettuce plant in step (a) to produce a seed;
(c) growing a progeny plant of a subsequent generation from the seed produced in step (b);
(d) crossing the progeny plant of a subsequent generation of step (c) with itself or the second Lettuce plant in step (a) to produce a Lettuce plant derived from the Lettuce cultivar VINDARA_13, VINDARA_16, and/or VINDARA_18.
15. The method of claim 14 further comprising the step of: (e) repeating step b) and/or c) for at least 1 more generation to produce a Lettuce plant derived from the Lettuce cultivar VINDARA_13, VINDARA_16, and/or VINDARA_18.
16. The method of claim 15, wherein said Lettuce plant derived from the Lettuce cultivar VINDARA_13, VINDARA_16, and/or VINDARA_18 produces with superior plant (canopy) diameter (cm), plant height (cm), number of leaves, and overall leaf area (cm²) as compared to traditional lettuce cultivars.
17. A method for producing an herbicide resistant Lettuce plant wherein the method comprises transforming the Lettuce plant of claim 7 with a transgene, wherein the transgene confers resistance to an herbicide selected from the group consisting of imidazolinone, sulfonylurea, glyphosate, glufosinate, L-phosphinothricin, triazine and benzonitrile.
18. An herbicide resistant Lettuce plant produced by the method of claim 17.
19. A method of producing an insect resistant Lettuce plant, wherein the method comprises transforming the Lettuce plant of claim 7 with a transgene that confers insect resistance.
20. An insect resistant Lettuce plant produced by the method of claim 19.
21. The Lettuce plant of claim 20, wherein the transgene encodes a Bacillus thuringiensis endotoxin.
22. A method of producing a disease resistant Lettuce plant wherein the method comprises transforming the Lettuce plant of claim 6 with a transgene that confers disease resistance.
23. A disease resistant Lettuce plant produced by the method of claim 22.
24. A method of producing a Lettuce plant with a value-added trait, wherein the method comprises transforming the Lettuce plant of claim 6 with a transgene encoding a protein selected from the group consisting of a ferritin, a nitrate reductase, and a monellin.
25. A Lettuce plant with a value-added trait produced by the method of claim 24.
26. A method of introducing a desired trait into Lettuce cultivar VINDARA_13, VINDARA_16, and/or VINDARA_18 wherein the method comprises:
a) crossing a VINDARA_13, VINDARA_16, and/or VINDARA_18 plant grown from VINDARA_13, VINDARA_16, and/or VINDARA_18 seed, wherein a representative sample of seed was deposited under Accession No. PTA-______, PTA-______ and/or PTA-______ with a plant of another Lettuce cultivar that comprises a desired trait to produce F₁ progeny plants, wherein the desired trait is selected from the group consisting of herbicide resistance, insect resistance, and resistance to bacterial disease, fungal disease, or viral disease;
b) selecting one or more progeny plants that have the desired trait to produce selected progeny plants;
c) crossing the selected progeny plants with the VINDARA_13, VINDARA_16, and/or VINDARA_18 plants to produce backcross progeny plants;
d) selecting for backcross progeny plants that have the desired trait and all of the physiological and morphological characteristics of Lettuce cultivar VINDARA_13, VINDARA_16, and/or VINDARA_18 listed in Table 1 to produce selected backcross progeny plants; and
e) repeating steps (c) and (d) three or more times in succession to produce selected fourth or higher backcross progeny plants that comprise the desired trait and all of the physiological and morphological characteristics of Lettuce cultivar VINDARA_13, VINDARA_16, and/or VINDARA_18.
27. A Lettuce plant produced by the method of claim 26, wherein the plant has the desired trait and all of the physiological and morphological characteristics of Lettuce cultivar VINDARA_13, VINDARA_16, and/or VINDARA_18.
28. The Lettuce plant of claim 27, wherein the desired trait is herbicide resistance and the resistance is conferred to an herbicide selected from the group consisting of imidazolinone, sulfonylurea, glyphosate, glufosinate, L-phosphinothricin, triazine and benzonitrile.
29. The Lettuce plant of claim 28, wherein the desired trait is insect resistance and the insect resistance is conferred by a transgene encoding a Bacillus thuringiensis endotoxin.
30. A method of producing a Lettuce plant with superior plant (canopy) diameter (cm), plant height (cm), number of leaves, and overall leaf area (cm2) comprising the steps of:
(a) crossing the plant of claim 7 with a second Lettuce plant to produce a progeny plant;
(b) crossing the progeny plant of step (a) with itself or the second Lettuce plant in step (a) to produce a seed;
(c) growing a progeny plant of a subsequent generation from the seed produced in step (b);
(d) crossing the progeny plant of a subsequent generation of step (c) with itself or the second Lettuce plant in step (a) to produce a Lettuce plant derived from the Lettuce VINDARA_13, VINDARA_16, and/or VINDARA_18 with superior plant (canopy) diameter (cm), plant height (cm), number of leaves, and overall leaf area (cm2).
31. A method for developing a Lettuce plant in a Lettuce plant breeding program, comprising applying plant breeding techniques comprising recurrent selection, backcrossing, pedigree breeding, marker enhanced selection, mutation breeding, or genetic modification to the Lettuce plant of claim 6, or its parts, to develop a Lettuce plant that produces superior plant (canopy) diameter (cm), plant height (cm), number of leaves, and overall leaf area (cm2).
32. A method of identifying a Lettuce plant for use in a plant breeding program comprising at least one allele associated with plant (canopy) diameter (cm), plant height (cm), number of leaves, and overall leaf area (cm2) in a Lettuce plant comprising:
a) genotyping at least one Lettuce plant with at least one nucleic acid marker selected from the group of SEQ ID NOs: 1-12; and
b) selecting based upon said genotyping at least one Lettuce plant comprising an allele of at least one of said nucleic acid markers that is associated with plant (canopy) diameter (cm), plant height (cm), number of leaves, and overall leaf area (cm2) for breeding.
33. The method according to claim 32, wherein the at least one Lettuce plant genotyped in step (a) and/or the at least one Lettuce plant selected in step (b) is a Lettuce plant from a population generated by a cross.
34. The method of claim 32, wherein said population is generated by a cross of at least one Lettuce plant having plant (canopy) diameter (cm), plant height (cm), number of leaves, and overall leaf area (cm2) with at least one Lettuce plant having no trait.
35. The method of claim 32, wherein said population is a segregating population.
36. The method of claim 32, wherein said cross is a backcross of at least one Lettuce plant having plant (canopy) diameter (cm), plant height (cm), number of leaves, and overall leaf area (cm2)ing with at least one Lettuce plant having no trait to introgress plant (canopy) diameter (cm), plant height (cm), number of leaves, and overall leaf area (cm2) into a Lettuce germplasm.
37. The method of claim 32 further comprising the step of crossing the Lettuce plant selected in step (b) to another Lettuce plant.
38. The method of claim 32, further comprising the step of obtaining seed from the Lettuce plant selected in step (b).
39. A Lettuce plant obtained by the method of claim 32, wherein said Lettuce plant comprises an allele of at least one nucleic acid molecule selected from SEQ ID NOs: 1-12 that is associated with plant (canopy) diameter (cm), plant height (cm), number of leaves, and overall leaf area (cm2), and produces the same traits.
40. A method of introgressing a plant (canopy) diameter (cm), plant height (cm), number of leaves, and overall leaf area (cm2) locus allele into a Lettuce plant, the method comprising the steps of:
a) crossing at least one first Lettuce plant comprising the plant (canopy) diameter (cm), plant height (cm), number of leaves, and overall leaf area (cm2) locus allele, wherein the allele comprises one or more of SEQ ID NOs: 1-12, with at least one second Lettuce plant in order to form a segregating population;
b) screening said segregating population with one or more nucleic acid markers to determine if one or more Lettuce plants contain the plant (canopy) diameter (cm), plant height (cm), number of leaves, and overall leaf area (cm2) locus allele comprising one or more of SEQ ID NOs: 1-12; and
c) selecting said plants based upon said screening from said segregating population one or more Lettuce plants comprising said plant (canopy) diameter (cm), plant height (cm), number of leaves, and overall leaf area (cm2) locus allele for further breeding.
41. The method according to claim 40, wherein at least one of the nucleic acid markers is located within 100 Kb of the plant (canopy) diameter (cm), plant height (cm), number of leaves, and overall leaf area (cm2) locus.
42. The method according to claim 40, wherein at least one of the nucleic acid markers is located within 5 cM of the plant (canopy) diameter (cm), plant height (cm), number of leaves, and overall leaf area (cm2) locus.
43. The method according to claim 40, wherein at least one of the nucleic acid markers exhibits a LOD score of greater than 2.0 with the plant (canopy) diameter (cm), plant height (cm), number of leaves, and overall leaf area (cm2) locus.
44. The method according to claim 40, wherein said population is generated by a cross of at least one Lettuce plant having plant (canopy) diameter (cm), plant height (cm), number of leaves, and overall leaf area (cm2) with at least one Lettuce plant having no trait.
45. A Lettuce plant obtained by the method of claim 40.
46. The Lettuce plant according to claim 45, wherein said Lettuce plant comprises an allele of at least one nucleic acid marker selected from the group of SEQ ID NOs: 1-12 that is associated with plant (canopy) diameter (cm), plant height (cm), number of leaves, and overall leaf area (cm2).
47. The Lettuce plant according to claim 45, wherein said Lettuce plant is homozygous for said allele.
48. The Lettuce plant of claim 45, wherein the Lettuce plant produces superior plant plant (canopy) diameter (cm), plant height (cm), number of leaves, and overall leaf area (cm2).
49. A method of creating a population of Lettuce plants each comprising at least one allele associated with plant (canopy) diameter (cm), plant height (cm), number of leaves, and overall leaf area (cm2), the method comprising the steps of:
a) genotyping a first population of Lettuce plants, said population or said plants containing at least one allele associated with plant (canopy) diameter (cm), plant height (cm), number of leaves, and overall leaf area (cm2), the at least one allele associated with plant (canopy) diameter (cm), plant height (cm), number of leaves, and overall leaf area (cm2) comprising one or more of SEQ ID NOs: 1-12;
b) selecting from said first population of Lettuce plants based upon said genotyping one or more Lettuce plants containing said at least one allele associated with plant (canopy) diameter (cm), plant height (cm), number of leaves, and overall leaf area (cm2); and
c) producing from said selected one or more Lettuce plants a second population of Lettuce plants comprising at least one allele associated with plant (canopy) diameter (cm), plant height (cm), number of leaves, and overall leaf area (cm2) comprising one or more of SEQ ID NO: 1-12.
50. The method of claim 49, wherein said producing comprises crossing the selected one or more Lettuce plants having the plant (canopy) diameter (cm), plant height (cm), number of leaves, and overall leaf area (cm2) allele with a Lettuce plant having no trait to produce a second population of Lettuce plants having plant (canopy) diameter (cm), plant height (cm), number of leaves, and overall leaf area (cm2), thereby creating a population of Lettuce plants comprising at least one allele associated with plant (canopy) diameter (cm), plant height (cm), number of leaves, and overall leaf area (cm2) comprising one or more of SEQ ID NOs: 1-12.
51. The method according to claim 49, wherein the first population of Lettuce plants genotyped in step (a) is a population generated by a cross.
52. The method according to claim 49, wherein said selected Lettuce plant(s) of step (b) produces superior plant (canopy) diameter (cm), plant height (cm), number of leaves, and overall leaf area (cm2).
53. The method of claim 49, wherein said first population of Lettuce plants is generated by a cross of at least one Lettuce plant having plant (canopy) diameter (cm), plant height (cm), number of leaves, and overall leaf area (cm2) with at least one Lettuce plant having no trait.
54. The method of claim 49, wherein said first population of Lettuce plants is a segregating population.
55. A population of Lettuce plants obtained by the method of claim 49, wherein the Lettuce plants produce superior plant (canopy) diameter (cm), plant height (cm), number of leaves, and overall leaf area (cm2).
56. An isolated nucleic acid molecule for detecting a molecular marker representing a polymorphism in Lettuce DNA, wherein said nucleic acid molecule comprises at least 15 nucleotides that include or are immediately adjacent to said polymorphism, wherein said nucleic acid molecule is at least 90% identical to a sequence of the same number of consecutive nucleotides in either strand of DNA that include or are immediately adjacent to said polymorphism, and wherein said molecular marker is selected from the group of SEQ ID NOs: 1-12.
57. The isolated nucleic acid molecule of claim 56, wherein the polymorphism is G at position 51 of SEQ ID NO:1, a G at position 51 of SEQ ID NO:2, a G at position 49 of SEQ ID NO:3, a G at position 49 of SEQ ID NO:4, a G at position 50 of SEQ ID NO:5, a G at position 50 of SEQ ID NO:6, a G at position 48 of SEQ ID NO;7, a T at position 50 of SEQ ID NO:8, A G at position 50 of SEQ ID NO:9, a G at position 51 of SEQ ID NO:10, a G at position 50 of SEQ ID NO: 11, or a C at position 49 of SEQ ID NO:12 as set forth in SEQ ID NOS 1-12.
58. An isolated nucleic acid molecule comprising SEQ ID NO: 1-12, or a nucleic acid sequence having at least 90% sequence identity to SEQ ID NO: 1-12 that includes G at position 51 of SEQ ID NO:1, a G at position 51 of SEQ ID NO:2, a G at position 49 of SEQ ID NO:3, a G at position 49 of SEQ ID NO:4, a G at position 50 of SEQ ID NO:5, a G at position 50 of SEQ ID NO:6, a G at position 48 of SEQ ID NO;7, a T at position 50 of SEQ ID NO:8, A G at position 50 of SEQ ID NO:9, a G at position 51 of SEQ ID NO:10, a G at position 50 of SEQ ID NO: 11, or a C at position 49 of SEQ ID NO:12.

### Examples

Vindara_13, Vindara_16, and Vindara_18 (Table 1) are dark green romaine or cos-type (Ryder, 1997) lettuce inbred lines (Lactuca sativa) that were developed Applicants as having superior plant (canopy) diameter (cm), plant height (cm), number of leaves, and overall leaf area (cm²) as compared to their maternal parents and F₁ hybrids grown under artificial conditions. Vindara_18 is and F₄ derived plant from a cross between LS00013 and LS01418. LS00013 was a single plant selection from the heterogenous plant introduction (PI) 251501 displaying a predominant stem-type growth architecture, while LS01418 was a single plant selection from the heterogenous PI 667711 with a predominant romaine-type architecture. Vindara_16 is an F₄ derived plant from a cross between LS00013 and LS00956. The LS00013 selection was the same single plant selection as previous described for Vindara_18, while LS00956 was a single plant selection from the heterogenous PI 612670 displaying a predominant romaine-type architecture. Vindara_13 is an F₄ derived plant from a cross between LS0040 and LS01418. LS0040 was a single plant selection from the heterogenous PI 342557 with a predominant butterhead-type architecture, while the LS01418 was the same single plant selection as previous described for Vindara_18. The single plant selections were made in the fall/winter of 2018/19 based on color, texture, and yield.

### Methods

Model Development. A global diversity collection consisting of >1,600 lettuce accessions was genotyped using a proprietary CIPHER platform. A training set consisting of 78 lines was selected for phenotypic discrimination under artificial growing conditions based on genotypic/phenotypic variant classes using a neural net. The training set was grown and evaluated for various agronomic characters. Prediction models were built using the neural net and a validation set consisting of 26 lines was selected and evaluated for the same agronomic characters as previously described for the training set. The data was used to refine the prediction models in the neural net and develop accelerated breeding algorithms.

Hybrid Development and Line Evaluations. Breeding simulations were performed using the neural net with the order constraints of: i) increase canopy diameter, ii) plant height, iii) number of leaves, iv) dark green color, and v) crispy texture. Forty-eight crossing pairs were suggested by the neural net based on the constraints and of these 23 were made in the field over the summer of 2019 at Aberdeen, Id. F₁ Hybrids from 20 of the crossing pairs, the parents, and selected commercial varieties were evaluated for agronomic characters under standardized artificial conditions at CO2 concentrations of 550 ppm and 1600 ppm over the Fall 2019. The evaluations were done using a completely randomized design with three replications for each entry. Analyses of Variance (ANOVA) was preformed using the genotype, replication, and genotype*replication for all traits within each of CO2 environments. No significant effects (P≤0.05) were observed for replicate or genotype*replication therefore all replicates were combined. A Tukey's Honestly Significant Difference (HSD) test (Abdi and Williams, 2010) was used to compare the means for each trait between the hybrids, parental lines, and selected cultivars.

Inbred Line Development. F₂ populations were derived from the best preforming hybrid lines which included: i) JAIS_0013, ii) JAIS_0016, and iii) JAIS_0018. Five F₁ hybrid plants for each line were allowed to self in 25:2's Crop Acceleration Node (CAN) to produce ≥200 seed for each population. The resulting F₂ seed were planted and genotyped at the seedling stage using trait CIPHERs for: i) Crispiness (N=3 loci), ii) Plant (canopy) Diameter (N=5 loci), and Plant Height (N=4 loci). F₂ plants with predicted traits below the selection threshold were culled and the remaining plants were selfed to produce F₃ seed for each line. The resulting seed were planted, Ciphered, culled, planted, and selfed to produce F₄ seed as previously described for the F₂ seed. The F₄ seed were then planted and ciphered to select the best lines from each of the populations.

The resulting lines, parents, and F₁ hybrids were evaluated for agronomic characters under standardized artificial conditions at CO2 concentration of 990 ppm over the Summer 2020. The evaluations were done using a completely randomized design with each RIL represented once and the parents and hybrids represented three times. The means and standard deviations for the RIL populations were compared to the corresponding parental lines and hybrids for each trait. The comparisons and predicted values from the CIPHER were used to select the best lines for derivation and propagation.

Hybrid Evaluation at 550 ppm CO₂ Environmental Comparisons (Table 2). The JAIS_L0016 hybrid had significantly greater leaf diameter (cm), fresh mass (g), number of leaves, and leaf area (cm²) than LS_00013 (maternal parent), LS_00956 (paternal parent), and both commercial cultivars. The JAIS_L0013 hybrid had significantly greater leaf diameter (cm), plant height (cm), number of leaves, and leaf area (cm²) than LS_00040 (maternal parent). In addition, it had significantly greater leaf diameter (cm), number of leaves, and fresh mass (g) than both commercial varieties. The only significant difference between JAIS_L0013 and LS_01418 (paternal parent) was for fresh mass, where LS_01418 was significant greater. No significant differences were observed between the lines for tip burn as measured by presence or absence on each leaf. Overall, JAIS_L0016 was similar to JAIS_L0013 for all traits measure with the exception of fresh mass (g) where JAIS_L0016 was significantly greater.

Hybrid Evaluation at 1600 ppm CO2 Environmental Comparisons (Table 3). The JAIS_L0016 hybrid had significantly greater canopy diameter (cm), fresh mass (g), and leaf area (cm²) than LS_00013 (maternal parent) and LS_00956 (paternal parent). In addition, JAIS_L0016 had significantly greater canopy diameter (cm) and leaf area (cm²) than both commercial varieties. The JAIS_L0013 hybrid had significantly greater leaf diameter (cm) and plant height (cm) than LS_00040 (maternal parent) and both commercial varieties. The JAIS_L0018 hybrid had significantly greater leaf area (cm²) than LS_00013 (maternal parent), LS_01418 (paternal parent) and significantly greater leaf diameter (cm), plant height (cm), fresh mass (g), number of leaves, and leaf area (cm²) than LS_00013 (maternal parent) and both the commercial varieties. Overall, JAIS_L0018 had significantly greater canopy diameter (cm), fresh mass (g), and leaf area (cm²) than JAIS_L0013. In addition, no significant differences were observed between the lines for tip burn as measured by presence or absence on each leaf.

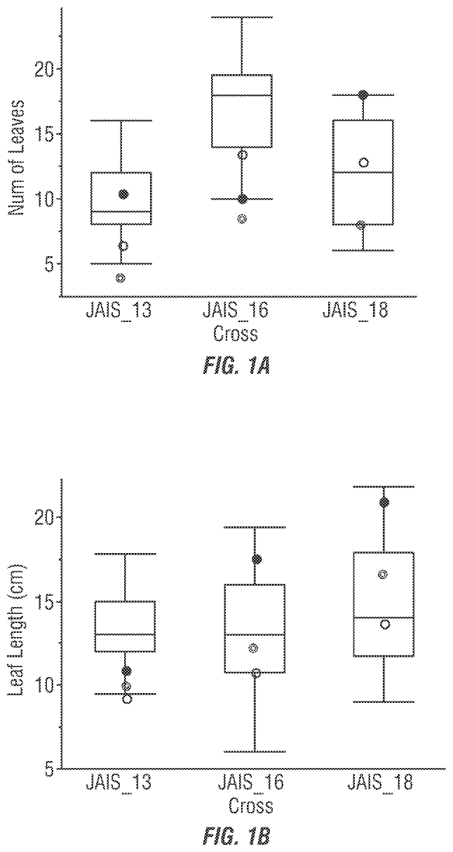

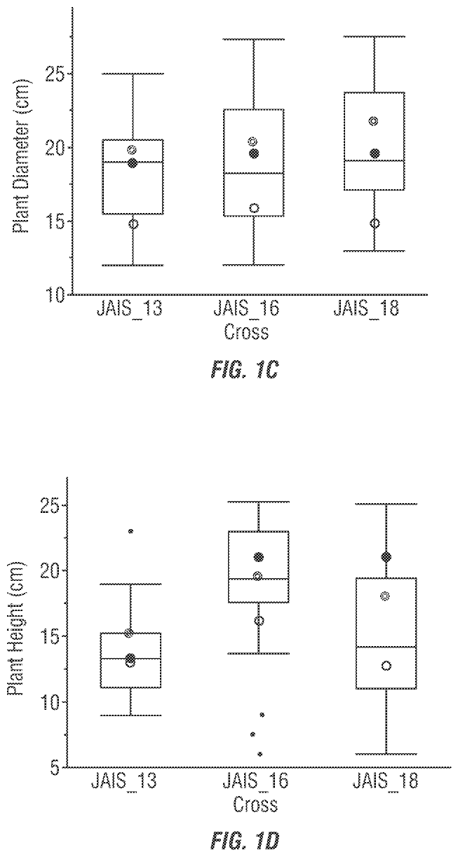

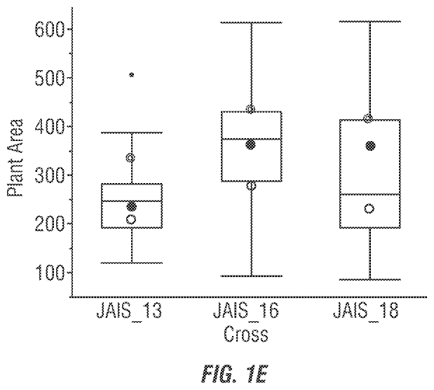

Inbred Evaluation at 990 ppm CO2 Environmental Comparisons (FIG. 1). The three populations were derived using 25:2's proprietary single-seed-descent approach assisted by CIPHER allele-based selection (Table 4). The resulting JAIS_0013 population consisted of 30 F₄ lines while the JAIS_0016 population consisted of 21 F₄ lines and the JAIS_0018 population consisted of 40 F₄ lines. RILs with superior trait measurements to the corresponding parental and hybrid lines were observed for all the populations. Overall, lines from the JAIS_0016 and JAIS_0018 preformed best, with JAIS_0016 having higher number of leaves and JAIS_0018 having longer leaves. Based on this evaluation, three lines from each of the JAIS_0016 and JAIS_0018 were selected for increase and further testing while two lines were selected from the JAIS_0013 populations. The best performing lines from each of these sub populations was selected for commercialization and be renamed as Vindara_13, Vindara_16, and Vindara_18.

### CONCLUSIONS

All the lettuce hybrids out preformed their parental lines and commercial varieties across all traits in both environments. JAIS_L0016 preformed best in the 550 ppm CO2 environment (Table 2), while JAIS_L0018 performed best in the 1600 ppm CO2 environment (Table 3). Although there were no significant differences for tip burn as measured by the presence or absence on each leaf, JAIS_L0013 had lowest incidence across both environments. Subsequently, RILs from each of the populations out performed their parental and hybrid lines in the 990 ppm CO2 environments (FIG. 4). The new Vindara_13, Vindara_16, and Vindara_18 lettuce varieties will have better yields with darker green color and firmer texture as compared to their parental and hybrid lines, as well as the commercial standards.

TABLE 1
Pedigree of Romaine Lettuce Hybrids (Lactuca sativa)

| Hybrid_ID | Maternal Parent | Paternal Parent |
|---|---|---|
| JAIS_L0018 | LS00013 (Selection | LS01418 (Selecction |
| | from PI251501) | from PI667711) |
| JAIS_L0016 | LS00013 (Selection | LS00956 (Selection |
| | from PI251501) | from PI612670) |
| JAIS_L0013 | LS00040 (Selection | LS01418 (Selecction |
| | from PI342557) | from PI667711) |

TABLE 2
Comparisons of Agronomic Characters Between Lettuce (Lactuca sativa)
Hybrids, Parents, and Commercial Varieties at 550 ppm CO2

| | Canopy Diameter | Plant Height | | Fresh Mass | | No. of Leaves | | Leaf Area | |
|---|---|---|---|---|---|---|---|---|---|
| Line_ID | (P < 0.005) | (P < 0.005) | | (P < 0.005) | | (P < 0.005) | | (P < 0.005) | Tip Burn |
| LS_01418 | 39.83 (A) | 16.50 | (A) | 156.68 | (B) | 29.50 | (BCD) | 2884.67 (A) | 2.00 |
| JAIS_L0016 | 44.20 (A) | 13.90 | (ABC) | 207.84 | (A) | 36 | (A) | 3890.32 (A) | 3.80 |
| JAIS_L0013 | 44.00 (A) | 15.17 | (ABC) | 101.36 | (C) | 29.00 | (AB) | 3490.63 (A) | 0.00 |
| LS_00956 | 23.83 (C) | 11.67 | (BCD) | 53.28 | (D) | 24.33 | (BCD) | 1243.74 (B) | 0.67 |
| LS_00040 | 21.83 (C) | 7.00 | (D) | 55.59 | (D) | 24.00 | (CD) | 1423.42 (B) | 1.33 |
| LS_00013 | 30.85 (B) | 12.58 | (BC) | 168.97 | (B) | 22.00 | (D) | 1938.92 (B) | 4.85 |
| Salad King | 29.17 (B) | 11.92 | (BCD) | 120.42 | (C) | 25.67 | (CD) | 1723.78 (B) | 0.17 |
| Foot Romaine | 32.33 (B) | 12.17 | (ABCD) | 120.42 | (C) | 25.67 | (CD) | 1968.54 (B) | 3.00 |

| | | | | | | |
|---|---|---|---|---|---|---|
| HSD (0.05) | 4.5 | 5.9 | 45.8 | 7.5 | 1005.2 | NS |

TABLE 3
Comparisons of Agronomic Characters Between Lettuce (Lactuca sativa)
Hybrids, Parents, and Commercial Varieties at 1600 ppm CO2

| | Canopy Diameter | | Plant Height | | Fresh Mass | | No. of Leaves | | Leaf Area | | |
|---|---|---|---|---|---|---|---|---|---|---|---|
| Line_ID | (P < 0.005) | | (P < 0.005) | | (P < 0.005) | | (P < 0.005) | | (P < 0.005) | | Tip Burn |
| JAIS_L0018 | 49.33 | (A) | 14.17 | (AB) | 280.07 | (A) | 39.00 | (A) | 3856.93 | (A) | 21.00 |
| LS_01418 | 47.00 | (AB) | 15.00 | (AB) | 229.30 | (ABC) | 36.00 | (A) | 2604.44 | (BCD) | 11.50 |
| JAIS_L0016 | 42.00 | (AB) | 14.00 | (ABC) | 237.25 | (AB) | 36.50 | (A) | 3414.19 | (AB) | 27.50 |
| JAIS_LS0013 | 41.33 | (B) | 17.50 | (A) | 200.37 | (BC) | 33.33 | (AB) | 1896.79 | (CD) | 15.30 |
| LS_00956 | 29.50 | (CD) | 10.50 | (CD) | 169.60 | (CD) | 28.00 | (B) | 1876.45 | (CD) | 13.00 |
| LS_00040 | 27.50 | (D) | 9.00 | (D) | 151.70 | (DE) | 32.00 | (AB) | 2249.67 | (CD) | 20.00 |
| LS_00013 | 27.00 | (D) | 9.50 | (D) | 135.00 | (E) | 29.50 | (B) | 2891.91 | (ABC) | 3.00 |
| Salad King | 31.00 | (CD) | 11.50 | (BCD) | 214.90 | (BC) | 30.00 | (B) | 2114.43 | (CD) | 18.50 |
| Foot Romaine | 30.33 | (CD) | 10.83 | (CD) | 167.60 | (CD) | 29.00 | (B) | 1815.91 | (D) | 19.00 |

| | | | | | | |
|---|---|---|---|---|---|---|
| HSD (0.05) | 7.5 | 3.6 | 65.2 | 7.2 | 1607.3 | NS |

TABLE 4
Genetic loci controlling key traits in lettuce with the correspondince effects on each

| | | | Linkage | | Effect |
|---|---|---|---|---|---|
| Trait | Locus | SNP* | group | Ref Sequence | (%) |
| Crispiness | | | | | |
| SEQ ID NO: 1 | NPGS_172 | A/G | not | CLS_S3_Contig1451-1-OP4 | 0.16 |
| | | | mapped | | |
| SEQ ID NO: 2 | NPGS_227 | A/G | 9 | CLS_S3_Contig9638-11-OP4 | 0.23 |
| SEQ ID NO: 3 | NPGS_281 | A/G | 2 | CLS_S3_Contig10032-2-OP5 | −0.17 |
| Plant Diameter | | | | | |
| SEQ ID NO: 4 | NPGS_128 | A/G | not | Contig821-6-OP1 | −0.23 |
| | | | mapped | | |
| SEQ ID NO: 5 | NPGS_136 | A/G | not | QGF20E14-1-OP1 | 0.26 |
| | | | mapped | | |
| SEQ ID NO: 6 | NPGS_177 | A/G | 9 | RHCLSX1795.b1_E18_1-OP3 | 0.29 |
| SEQ ID NO: 7 | NPGS_190 | C/G | 8 | CLX_S3_Contig746_1610_1-OP2 | −0.17 |
| SEQ ID NO: 8 | NPGS_95 | A/T | 8 | Contig6808-1-OP1 | −0.17 |
| Plant Height | | | | | |
| SEQ ID NO: 9 | NPGS_106 | A/G | 8 | CLS_S3_Contig8617-1-OP5 | −0.72 |
| SEQ ID NO: 10 | NPGS_122 | A/G | 1 | RHCLS_S3_Contig2870_5-OP3 | 1.27 |
| SEQ ID NO: 11 | NPGS_151 | A/G | not | CLS_S3_Contig4373-4-OP4 | −1.25 |
| | | | mapped | | |
| SEQ ID NO: 12 | NPGS_47 | A/C | not | CLS_S3_Contig4572-1-OP4 | 0.82 |
| | | | mapped | | |

Context sequence with SNP* Marker SNP (underlined) is present in SEQ ID NO

SEQ ID NO: 1
AGCTGAAGTGGNGGAGAATGAATGGTTTAAGAAAGGATATGTGCCACCT
AGATTTGAACA[A/G]GAGGATGTTAGTCTTGCTGATGTGGATGCTATT
TTCAATGAAGCNGGGGATTCTCCTATC
SEQ ID NO: 2
GAACACACATANTNAAGNCAAGAAATTGACAATGAAAATGAACTTCATG
AACTCTCTAGA[A/G]CCCCANATGGGTTCTAGAAGCTTCCCAATGAAA
AGAAGACCNATTGTGCNGCTAAGAACC
SEQ ID NO: 3
GATATCTGTAGNGTCAACCATTTGTTCACCNTNTNCCATCAATATTGGC
ACCTTCTTGTA[A/G]TCAGACCATTTGATTTCCTTTTTGTTGATGGGA
TTGACTTCCACAATTTTGTATGGNATA
SEQ ID NO: 4
AAATCTATTCATCTATGTTTGCATTCTAATTTAGAAGTAGACTCCCTGC
ATTTGATTTGC[A/G]TGACTTCTCAAATAGTGTCAACTAATGCCAAAA
GGGTATGTGCACAATCAGCATGACTTA
SEQ ID NO: 5
TTCTGTGGATGGCCACACATTTTGGGTAACGAAGGCCCACAGAATTCCG
GATTCCCTCC[A/G]GTGAAGTCTCAAATATACTGAACTGTGTTCCTG
ATGGAATACGTCCAACAAGATGGTTT
SEQ ID NO: 6
AGGGAGAAGGAGAGGGACCTTGTGGGTTTTCCATGAGAGGAACTGAGCT
TGTGTCAATGG[A/G]ATGGAGGAACTTGGACCTGAGTTCTTGTTCGCT
TCGTCTTCTTGATGTTGTTGTTCTTCT
SEQ ID NO: 7
GATGATTTCTACTTTCATTCCTAACCGTTGGATGTGATATCTTGATACC
TCCGCCCGTAC[C/G]GGTCTCGGGTCGACAATGATCGCTTTCATGCCA
TGAAACTCTGATTTTGACTGGCCGTTG
11
SEQ ID NO: 8
TAACATAACAAATAATTTTTTGCTATGAATGAATACTTGAGGRAAATGA
GTGATTTTTAT[A/T]CAGAATGAGAGAAAGAGGAGAACCTGACAAAGA
TTGTAAGTGAAGCTGTCATAATTGTCA
SEQ ID NO: 9
NCATCCNTTCCAAGGAGTCCAAACTAAACTNTGAAGCTTGGTTAAGTGG
NTACCATGGTC[A/G]AATTCCCCCTCTTCAGGAGGNACTAAATCGTCN
TTCCACCATTCCAAATTCATCATNTNA
SEQ ID NO: 10
AGTCAACCCCATCATCCNAMACATNTGGMRAAATACAAATGTCAACAAC
CAACAACTTCC[A/G]AAATGGTGAAGTTTCGATTGATCGTTTTGATCT
TCATCTGGGTTCGGAATGCAACGAAGA
SEQ ID NO: 11
TAACTNGGAATATNGTTGAATNANTACGGANGCATANTGTTCGGGTGTA
AACTGTCGGCT[A/G]GTNTGGTAGTAATTAGGCATGAAATANTTATTA
AGGTAATCATTGNTTCCTAATCCAACT
SEQ ID NO: 12
AAAGCTCTATCACCCCTTCTCAAATATAAAAGGATTTGGACTCCCTGTT
AAATTTCTCAT[A/C]ATTGCCACCAGTCGTTCATCCATATCATCAATC
TCTGGAAGAAACTTGAGAGCATGATAG
*SNP allele = dominant (wildtype) over recessive (D/R) with the dominant allele conditioning the effect.

### LITERATURE

Abdi, H., Williams, L. J., 2010. Tukey's Honestly Significant Difference (HSD) test. Encyclopedia of Research Design. Sage, Thousand Oaks, Calif. 1-5.
Ryder, E. J. 1997. Origin and history of lettuce, types of lettuce, and production. Pages 1-8 in: Compendium of Lettuce Diseases. R. M. Davis, K. V. Subbarao, R. N. Raid, and E. A. Kurtz, eds. American Phytopathological Society, St. Paul, Minn.

### Deposits

Applicant(s) will make a deposit of at least 625 seeds of Lettuce Cultivars Vindara 13, 16, and 18 with the American Type Culture Collection (ATCC), Manassas, Va. 20110 USA, ATCC Deposit Nos. ______, and ______. The seeds deposited with the ATCC will be taken from the deposit maintained by Vindara at 815 S 1st Ave Suite A, Pocatello, Id., 83201 since prior to the filing date of this application. Access to this deposit will be available during the pendency of the application to the Commissioner of Patents and Trademarks and persons determined by the Commissioner to be entitled thereto upon request. Upon issue of claims, the Applicant(s) will make available to the public, pursuant to 37 CFR 1.808, a deposit of at least 625 seeds of cultivars Vindara 13, 16, and 18 with the American Type Culture Collection (ATCC), 10801 University Boulevard, Manassas, Va. 20110-2209. This deposit of the lettuce cultivars Vindara 13, 16, and 18 will be maintained in the ATCC depository, which is a public depository, for a period of 30 years, or 5 years after the most recent request, or for the enforceable life of the patent, whichever is longer, and will be replaced if it becomes nonviable during that period.

Additionally, Applicants have or will satisfy all the requirements of 37 C.F.R. §§ 1.801-1.809, including providing an indication of the viability of the sample. Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

What is claimed is:

1. A Lettuce cultivar with superior plant (canopy) diameter (cm), plant height (cm), number of leaves, and overall leaf area (cm²) as compared to traditional lettuce cultivars and which includes one or more of SEQ ID NO: 1-12.

2. A plant of the lettuce cultivar of claim 1.

3. The Lettuce cultivar of claim 1, wherein the cultivar has the cultivar of VINDARA_13, VINDARA_16, and/or VINDARA_18 as an ancestor.

4. A Lettuce cultivar designated VINDARA_13, VINDARA_16, and/or VINDARA_18, wherein a representative sample of seed of said cultivar was deposited under Accession No. PTA-______, PTA-______ or PTA-______.

5. Seed of Lettuce cultivar designated VINDARA_13, VINDARA_16, and/or VINDARA_18, wherein a representative sample of seed of said cultivar was deposited under Accession No. PTA-______, PTA-______ or PTA-______.

6. A Lettuce plant, or a part thereof, produced by growing the seed of claim 6.

7. A tissue culture of cells produced from the plant of claim 7, wherein said cells of the tissue culture are produced from a plant part selected from the group consisting of embryo, meristematic cell, leaf, cotyledon, hypocotyl, stem, root, root tip, pistil, anther, flower, seed and pollen.

8. A protoplast produced from the plant of claim 7.

9. A protoplast produced from the tissue culture of claim 8.

10. A Lettuce plant regenerated from the tissue culture of claim 10, wherein the plant has all of the morphological and physiological characteristics of cultivar VINDARA_13, VINDARA_16, and/or VINDARA_18, wherein a representative sample of seed was deposited under Accession No. PTA-______, PTA-______ or PTA-______.

11. A method for producing a hybrid Lettuce seed, wherein the method comprises: crossing the Lettuce plant of claim 1 with a different Lettuce plant and harvesting the resultant F₁ hybrid Lettuce seed.

12. A hybrid Lettuce seed produced by the method of claim 11.

13. A hybrid Lettuce plant, or a part thereof, produced by growing said hybrid seed of claim 11.

14. A method of producing a Lettuce plant derived from the Lettuce cultivar VINDARA_13, VINDARA_16, and/or VINDARA_18 wherein the method comprises:
(a) crossing the plant of claim 7 with a second Lettuce plant to produce a progeny plant;
(b) crossing the progeny plant of step (a) with itself or the second Lettuce plant in step (a) to produce a seed;
(c) growing a progeny plant of a subsequent generation from the seed produced in step (b);
(d) crossing the progeny plant of a subsequent generation of step (c) with itself or the second Lettuce plant in step (a) to produce a Lettuce plant derived from the Lettuce cultivar VINDARA_13, VINDARA_16, and/or VINDARA_18.

15. The method of claim 14 further comprising the step of: (e) repeating step b) and/or c) for at least 1 more generation to produce a Lettuce plant derived from the Lettuce cultivar VINDARA_13, VINDARA_16, and/or VINDARA_18.

16. The method of claim 15, wherein said Lettuce plant derived from the Lettuce cultivar VINDARA_13, VINDARA_16, and/or VINDARA_18 produces with superior plant (canopy) diameter (cm), plant height (cm), number of leaves, and overall leaf area (cm²) as compared to traditional lettuce cultivars.

17. A method for producing an herbicide resistant Lettuce plant wherein the method comprises transforming the Lettuce plant of claim 7 with a transgene, wherein the transgene confers resistance to an herbicide selected from the group consisting of imidazolinone, sulfonylurea, glyphosate, glufosinate, L-phosphinothricin, triazine and benzonitrile.

18. An herbicide resistant Lettuce plant produced by the method of claim 17.

19. A method of producing an insect resistant Lettuce plant, wherein the method comprises transforming the Lettuce plant of claim 7 with a transgene that confers insect resistance.

20. An insect resistant Lettuce plant produced by the method of claim 19.

21. The Lettuce plant of claim 20, wherein the transgene encodes a Bacillus thuringiensis endotoxin.

22. A method of producing a disease resistant Lettuce plant wherein the method comprises transforming the Lettuce plant of claim 6 with a transgene that confers disease resistance.

23. A disease resistant Lettuce plant produced by the method of claim 22.

24. A method of producing a Lettuce plant with a value-added trait, wherein the method comprises transforming the Lettuce plant of claim 6 with a transgene encoding a protein selected from the group consisting of a ferritin, a nitrate reductase, and a monellin.

25. A Lettuce plant with a value-added trait produced by the method of claim 24.

26. A method of introducing a desired trait into Lettuce cultivar VINDARA_13, VINDARA_16, and/or VINDARA_18 wherein the method comprises:
a) crossing a VINDARA_13, VINDARA_16, and/or VINDARA_18 plant grown from VINDARA_13, VINDARA_16, and/or VINDARA_18 seed, wherein a representative sample of seed was deposited under Accession No. PTA-______, PTA-______ and/or PTA-______ with a plant of another Lettuce cultivar that comprises a desired trait to produce F₁ progeny plants, wherein the desired trait is selected from the group consisting of herbicide resistance, insect resistance, and resistance to bacterial disease, fungal disease, or viral disease;
b) selecting one or more progeny plants that have the desired trait to produce selected progeny plants;
c) crossing the selected progeny plants with the VINDARA_13, VINDARA_16, and/or VINDARA_18 plants to produce backcross progeny plants;
d) selecting for backcross progeny plants that have the desired trait and all of the physiological and morphological characteristics of Lettuce cultivar VINDARA_13, VINDARA_16, and/or VINDARA_18 listed in Table 1 to produce selected backcross progeny plants; and
e) repeating steps (c) and (d) three or more times in succession to produce selected fourth or higher backcross progeny plants that comprise the desired trait and all of the physiological and morphological characteristics of Lettuce cultivar VINDARA_13, VINDARA_16, and/or VINDARA_18.

27. A Lettuce plant produced by the method of claim 26, wherein the plant has the desired trait and all of the physiological and morphological characteristics of Lettuce cultivar VINDARA_13, VINDARA_16, and/or VINDARA_18.

28. The Lettuce plant of claim 27, wherein the desired trait is herbicide resistance and the resistance is conferred to an herbicide selected from the group consisting of imidazolinone, sulfonylurea, glyphosate, glufosinate, L-phosphinothricin, triazine and benzonitrile.

29. The Lettuce plant of claim 28, wherein the desired trait is insect resistance and the insect resistance is conferred by a transgene encoding a Bacillus thuringiensis endotoxin.

30. A method of producing a Lettuce plant with superior plant (canopy) diameter (cm), plant height (cm), number of leaves, and overall leaf area (cm2) comprising the steps of:
(a) crossing the plant of claim 7 with a second Lettuce plant to produce a progeny plant;
(b) crossing the progeny plant of step (a) with itself or the second Lettuce plant in step (a) to produce a seed;
(c) growing a progeny plant of a subsequent generation from the seed produced in step (b);
(d) crossing the progeny plant of a subsequent generation of step (c) with itself or the second Lettuce plant in step (a) to produce a Lettuce plant derived from the Lettuce VINDARA_13, VINDARA_16, and/or VINDARA_18 with superior plant (canopy) diameter (cm), plant height (cm), number of leaves, and overall leaf area (cm2).

31. A method for developing a Lettuce plant in a Lettuce plant breeding program, comprising applying plant breeding techniques comprising recurrent selection, backcrossing, pedigree breeding, marker enhanced selection, mutation breeding, or genetic modification to the Lettuce plant of claim 6, or its parts, to develop a Lettuce plant that produces superior plant (canopy) diameter (cm), plant height (cm), number of leaves, and overall leaf area (cm2).

32. A method of identifying a Lettuce plant for use in a plant breeding program comprising at least one allele associated with plant (canopy) diameter (cm), plant height (cm), number of leaves, and overall leaf area (cm2) in a Lettuce plant comprising:
a) genotyping at least one Lettuce plant with at least one nucleic acid marker selected from the group of SEQ ID NOs: 1-12; and
b) selecting based upon said genotyping at least one Lettuce plant comprising an allele of at least one of said nucleic acid markers that is associated with plant (canopy) diameter (cm), plant height (cm), number of leaves, and overall leaf area (cm2) for breeding.

33. The method according to claim 32, wherein the at least one Lettuce plant genotyped in step (a) and/or the at least one Lettuce plant selected in step (b) is a Lettuce plant from a population generated by a cross.

34. The method of claim 32, wherein said population is generated by a cross of at least one Lettuce plant having plant (canopy) diameter (cm), plant height (cm), number of leaves, and overall leaf area (cm2) with at least one Lettuce plant having no trait.

35. The method of claim 32, wherein said population is a segregating population.

36. The method of claim 32, wherein said cross is a backcross of at least one Lettuce plant having plant (canopy) diameter (cm), plant height (cm), number of leaves, and overall leaf area (cm2)ing with at least one Lettuce plant having no trait to introgress plant (canopy) diameter (cm), plant height (cm), number of leaves, and overall leaf area (cm2) into a Lettuce germplasm.

37. The method of claim 32 further comprising the step of crossing the Lettuce plant selected in step (b) to another Lettuce plant.

38. The method of claim 32, further comprising the step of obtaining seed from the Lettuce plant selected in step (b).

39. A Lettuce plant obtained by the method of claim 32, wherein said Lettuce plant comprises an allele of at least one nucleic acid molecule selected from SEQ ID NOs: 1-12 that is associated with plant (canopy) diameter (cm), plant height (cm), number of leaves, and overall leaf area (cm2), and produces the same traits.

40. A method of introgressing a plant (canopy) diameter (cm), plant height (cm), number of leaves, and overall leaf area (cm2) locus allele into a Lettuce plant, the method comprising the steps of:
a) crossing at least one first Lettuce plant comprising the plant (canopy) diameter (cm), plant height (cm), number of leaves, and overall leaf area (cm2) locus allele, wherein the allele comprises one or more of SEQ ID NOs: 1-12, with at least one second Lettuce plant in order to form a segregating population;
b) screening said segregating population with one or more nucleic acid markers to determine if one or more Lettuce plants contain the plant (canopy) diameter (cm), plant height (cm), number of leaves, and overall leaf area (cm2) locus allele comprising one or more of SEQ ID NOs: 1-12; and
c) selecting said plants based upon said screening from said segregating population one or more Lettuce plants comprising said plant (canopy) diameter (cm), plant height (cm), number of leaves, and overall leaf area (cm2) locus allele for further breeding.
